(19) United States
(12) Patent Application Publication
(10) Pub. No.: US 2025/0116663 A1
(43) Pub. Date: Apr. 10, 2025

(54) ANALYSIS SUBSTRATE, ANALYSIS METHOD, ANALYSIS SYSTEM, AND ANALYSIS APPARATUS

(71) Applicants: CANON MEDICAL SYSTEMS CORPORATION, Tochigi (JP); The General Hospital Corporation, Boston, MA (US)

(72) Inventors: Kohei WATANABE, Utsunomiya (JP); Noriyuki KASAGI, Utsunomiya (JP); Satoru KOMATSU, Melrose, MA (US); Tomoki FUJITO, Nasushiobara (JP); Hyungsoon IM, Weston, MA (US)

(73) Assignees: CANON MEDICAL SYSTEMS CORPORATION, Tochigi (JP); The General Hospital Corporation, Boston, MA (US)

(21) Appl. No.: 18/907,183

(22) Filed: Oct. 4, 2024

Related U.S. Application Data
(60) Provisional application No. 63/588,411, filed on Oct. 6, 2023.

(51) Int. Cl. G01N 33/543; G01N 21/64; G01N 33/58

(52) U.S. Cl. CPC G01N 33/54373; G01N 21/6428; G01N 33/582; G01N 2021/6441

(57) ABSTRACT

An analysis substrate according to an embodiment is an analysis substrate for performing analysis on fine particles, and includes a plurality of capture regions that capture the fine particles; and a plurality of first isolators whose wettability changes in response to a stimulus, the plurality of first isolators being provided along a first direction between the capture regions.

### CROSS REFERENCE TO RELATED APPLICATIONS

This application is based upon and claims the benefit of priority from the U.S. Provisional Patent Application No. 63/588,411, filed on Oct. 6, 2023, the entire contents of which are incorporated herein by reference.

### FIELD

Embodiments disclosed in the present specification and the drawings relate to an analysis substrate, an analysis method, an analysis system, and an analysis apparatus.

### BACKGROUND

In the related art, a liquid biopsy, which is an examination that performs a disease diagnose and the like using a body fluid such as blood, has been performed as an examination with a low burden on a subject such as a patient. In the liquid biopsy, a technique for determining the presence or absence of a disease and the type of the disease based on a detection result of a biomarker such as protein or gene is known.

In recent years, extracellular vesicles (hereinafter, also referred to as EVs, as necessary) released from cells or tissues have attracted attention as biomarkers used for a liquid biopsy, and various techniques for analyzing EVs have been developed. For example, in the analysis of EVs, there is a single EV analysis in which each particle is analyzed from the viewpoint of high sensitivity, and as one method of the single EV analysis, there is a method of capturing EVs on a substrate and performing imaging. In cancer diagnosis performed using such an analysis method, it is common to analyze a plurality of biomarkers. Therefore, a method in which biomarkers of EVs captured on a substrate are subjected to multiple stain with antibodies to analyze a plurality of biomarkers on EVs has been proposed.

However, the number of EVs captured in one capture region that can be multiplexed is usually limited to about four colors since it is limited by the number of fluorescent dyes used for staining or the number of light sources and filters. In addition, in detection of a fluorescence signal of a minute EV, a plasmon fluorescence enhancement method for enhancing the fluorescence signal of the minute EV using a plasmon substrate is known, but the plasmon fluorescence enhancement method has a narrow wavelength range that can be enhanced, and thus has a problem of multiplexing. Furthermore, these problems similarly occur in analysis of fine particles such as viruses, bacteria, liposomes other than EVs. Therefore, in the case of analyzing a plurality of biomarkers, it is desired to improve analysis efficiency and improve a throughput of fine particle analysis by simultaneously analyzing a plurality of biomarkers on one substrate.

### BRIEF DESCRIPTION OF THE DRAWINGS

FIG. 1 is a view for explaining an outline of a liquid biopsy.

FIG. 2 is a block diagram illustrating an example of a configuration of an analysis system according to a first embodiment.

FIG. 3 is a top view illustrating an example of an analysis substrate according to the first embodiment.

FIG. 4 is a cross-sectional view taken along ling A-A in FIG. 3.

FIG. 5 is a flowchart for explaining contents of analysis processing performed by an analysis apparatus according to the first embodiment.

FIG. 6 is a view illustrating an example of an analysis substrate arranged in the analysis apparatus in the first embodiment.

FIG. 7 is a view illustrating an example of an analysis substrate in which a stimulus is applied to a flow path former by the analysis apparatus according to the first embodiment.

FIG. 8 is a view illustrating an example of an analysis substrate onto which a specimen is introduced in the first embodiment.

FIG. 9 is a view illustrating an example of an analysis substrate on which the specimen is cleaned in the first embodiment.

FIG. 10 is a view illustrating an example of an analysis substrate in which a stimulus is applied to a plurality of first isolators by the analysis apparatus according to the first embodiment.

FIG. 11 is a view illustrating an example of an analysis substrate onto which a labeling reagent is introduced in the first embodiment.

FIG. 12 is a view illustrating an example of an analysis substrate on which the labeling reagent is cleaned in the first embodiment.

FIG. 13 is a top view illustrating an example of an analysis substrate according to a second embodiment.

FIG. 14 is a cross-sectional view taken along ling B-B in FIG. 13.

FIG. 15 is a flowchart for explaining contents of analysis processing performed by an analysis apparatus according to the second embodiment.

FIG. 16 is a view illustrating an example of an analysis substrate arranged in the analysis apparatus in the second embodiment.

FIG. 17 is a view illustrating an example of an analysis substrate in which a stimulus is applied to a plurality of second isolators by the analysis apparatus according to the second embodiment.

FIG. 18 is a view illustrating an example of an analysis substrate onto which a specimen is introduced in the second embodiment.

FIG. 19 is a view illustrating an example of an analysis substrate on which the specimen is cleaned in the second embodiment.

FIG. 20 is a view illustrating an example of an analysis substrate in which a stimulus is applied to a plurality of first isolators by the analysis apparatus according to the second embodiment.

FIG. 21 is a view illustrating an example of a analysis substrate onto which a labeling reagent is introduced in the second embodiment.

FIG. 22 is a view illustrating an example of an analysis substrate on which the labeling reagent is cleaned in the second embodiment.

FIG. 23 is a block diagram illustrating an example of a configuration of an analysis system according to a third embodiment.

FIG. 24 is a top view illustrating an example of an analysis substrate according to a third embodiment.

FIG. 25 is a cross-sectional view taken along ling C-C in FIG. 24.

FIG. 26 is a flowchart for explaining contents of analysis processing performed by an analysis apparatus according to the third embodiment.

FIG. 27 is a view illustrating a state where an analysis apparatus radiates excitation light to an analysis substrate in the third embodiment.

FIG. 28 is a top view illustrating an example of an analysis substrate according to a fourth embodiment.

FIG. 29 is a perspective view illustrating an example of an analysis substrate according to the fourth embodiment.

FIG. 30 is a view illustrating an example of an analysis substrate onto which a specimen is introduced in the fourth embodiment.

FIG. 31 is a top view illustrating an example of an analysis substrate according to a fifth embodiment.

FIG. 32 is a perspective view illustrating an example of a analysis substrate according to the fifth embodiment.

FIG. 33 is a view illustrating an example of an analysis substrate onto which a specimen is introduced in the fifth embodiment.

FIG. 34 is a block diagram illustrating an example of a configuration of an analysis system according to a sixth embodiment.

FIG. 35 is a top view illustrating an example of an analysis substrate according to a sixth embodiment.

FIG. 36 is a perspective view illustrating an example of a separator according to the sixth embodiment.

FIG. 37 is a flowchart for explaining contents of analysis processing performed by an analysis apparatus according to the sixth embodiment.

FIG. 38 is a view for explaining a positional relationship between the separator and the analysis substrate at the time of introducing the specimen in the sixth embodiment.

FIG. 39 is a view illustrating a state where the separator is arranged on the analysis substrate at the time of introducing the specimen in the sixth embodiment.

FIG. 40 is a view illustrating a positional relationship between the separator and the analysis substrate at the time of introducing a labeling reagent in the sixth embodiment.

FIG. 41 is a view illustrating a state where the separator is arranged on the analysis substrate at the time of introducing the labeling reagent in the fifth embodiment.

FIG. 42 is a view illustrating another example of a separator according to the sixth embodiment.

FIG. 43 is a view illustrating an example of a configuration of an analysis system according to a first modification.

FIG. 44 is a top view illustrating an example of a configuration of an analysis substrate according to the first modification.

FIG. 45 is a flowchart for explaining contents of analysis processing performed by an analysis apparatus according to the first modification.

FIG. 46 is a view illustrating an example of an analysis substrate onto which a specimen is introduced in an analysis system according to a modification.

### DETAILED DESCRIPTION

Hereinafter, embodiments of an analysis substrate, an analysis method, an analysis system, and an analysis apparatus will be described with reference to the drawings. Note that, in the following description, components having substantially the same functions and configurations are denoted by the same reference numerals, and redundant description will be given only when necessary.

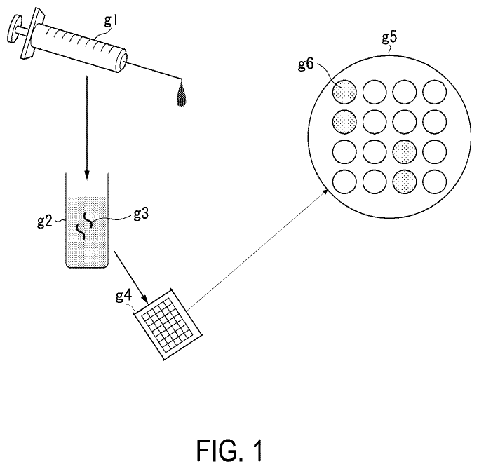

FIG. 1 is a view for explaining an outline of a liquid biopsy. As illustrated in FIG. 1, for example, first, a doctor collects blood from a subject using a syringe g1. The blood collected from the subject contains, for example, various tumor-derived components. The tumor-derived component g3 is, for example, EVs, circulating tumor RNA (tDNA), circulating tumor DNA (ctDNA), tumor proteins, circulating tumor cells (CTCs), platelets educated by tumor (TEPs), or the like.

In the liquid biopsy, a specimen containing EVs is introduced onto an analysis substrate g4. Then, EVs extracted from the specimen are labeled with red fluorescent dyes (g2 and g3). Next, in the liquid biopsy, the cleaned analysis substrate is imaged by an imaging apparatus (g5), and a signal is detected. The signal is, for example, the presence or absence of expression of red g6 (represented by the hatched circle in FIG. 1), the number of expressions, or the like. As a result, in the liquid biopsy, for example, it is possible to detect that there is cancer onset or a suspicion of a disease such as a tumor.

Note that, in the example illustrated in FIG. 1, the specimen used for a liquid biopsy has been described as blood, but the present invention is not limited to blood. For example, a body fluid other than blood such as saliva may be used as the specimen.

### First Embodiment

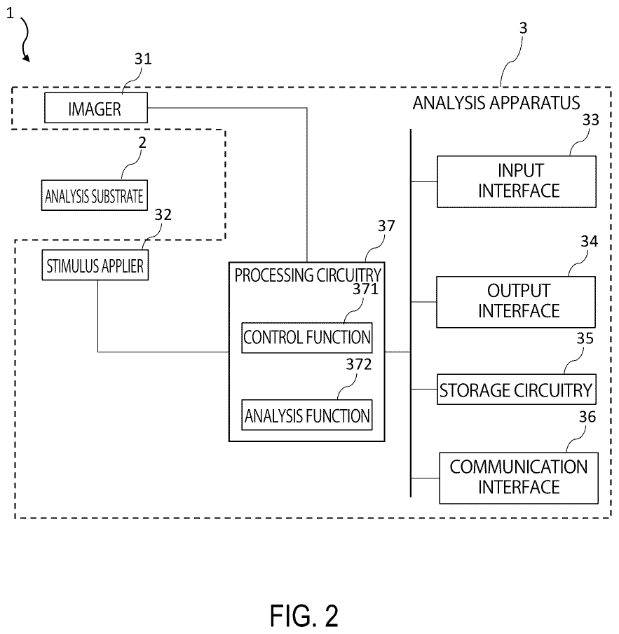

Next, an example of a configuration of an analysis system according to a first embodiment will be described with reference to FIGS. 2 to 4. FIG. 2 is a block diagram illustrating an example of a configuration of the analysis system according to the first embodiment. As illustrated in FIG. 2, an analysis system 1 according to the present embodiment includes an analysis substrate 2 and an analysis apparatus 3.

The analysis substrate 2 is a substrate for performing analysis on fine particles, and is a substrate arranged in the analysis apparatus 3. The analysis substrate 2 is detachably arranged in the analysis apparatus 3.

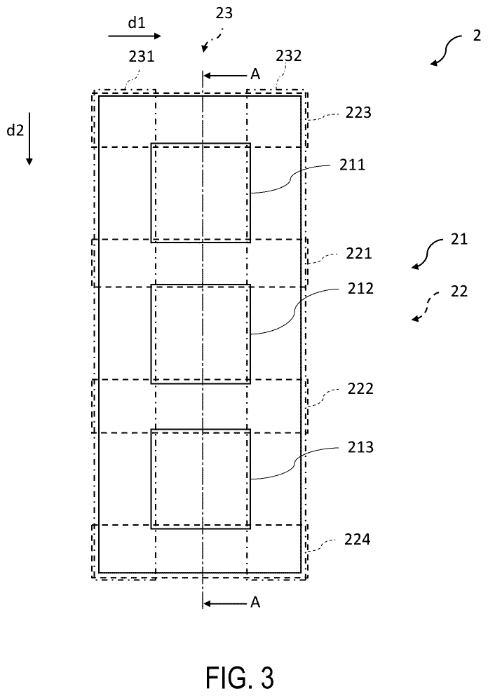

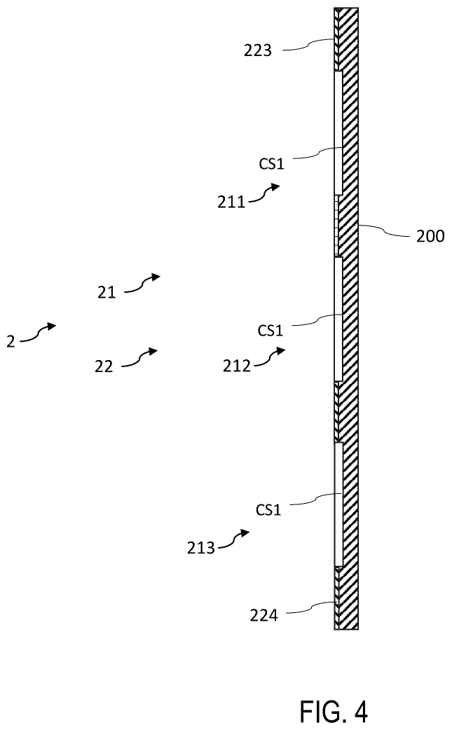

The detailed configuration of the analysis substrate 2 will be described in detail with reference to FIGS. 3 and 4. FIG. 3 is a top view illustrating an example of the analysis substrate according to the first embodiment. FIG. 4 is a cross-sectional view taken along ling A-A in FIG. 3. As illustrated in FIGS. 3 and 4, the analysis substrate 2 according to the present embodiment includes a plurality of capture regions 21, a plurality of first isolators 22, a flow path former 23, and a substrate body 200. Here, the fine particles include EVs, nano-micelles, viruses, bacteria, and the like. In the following description, an embodiment will be described by taking a case where the fine particles are EVs as an example.

The plurality of capture regions 21 are regions that capture fine particles. The plurality of capture regions 21 according to the present embodiment are regions that capture EVs. As illustrated in FIG. 3, in the present embodiment, three capture regions 211, 212, and 213 are provided at a predetermined interval along a second direction d2. In the example illustrated in FIG. 3, the three capture regions 211, 212, and 213 have a rectangular shape. In addition, as illustrated in FIG. 4, the plurality of capture regions 21 according to the present embodiment are formed to be recessed from a surface of the substrate body 200. Surfaces of the plurality of capture regions 21 according to the present embodiment are surfaces capable of covalently bonding to fine particles. Specifically, polyethylene glycol (PEG) is bonded to capture surfaces CS1, which are the surfaces of the plurality of capture regions 21 according to the present embodiment, via N-hydroxysuccinimide (NHS). In the present embodiment, the EVs are fixed to the capture surfaces CS1 of the plurality of capture regions 21 on the substrate by fixing the EVs contained in the specimen in which polyethylene glycol (PEG) is introduced into the plurality of capture regions 21.

Note that, in the example illustrated in FIG. 3, three capture regions are provided on the substrate body 200 in one direction, but the number of the plurality of capture regions 21 provided on the substrate body 200 is not limited to three. That is, the number of the plurality of capture regions 21 is arbitrary, and two capture regions may be provided in one direction, or four or more capture regions may be provided.

A plurality of first isolators 22 are provided along a first direction d1 between the capture regions, and are sections whose wettability changes in response to a stimulus. As illustrated in FIG. 3, each of the plurality of first isolators 22 according to the present embodiment is a region surrounded by the broken line on the substrate body 200, and includes a first isolator 221 provided along the first direction d1 between the capture region 211 and the capture region 212, and a first isolator 222 provided along the first direction d1 between the capture region 212 and the capture region 213. In addition, as illustrated in FIG. 3, each of the plurality of first isolators 22 according to the present embodiment includes a first isolator 223 provided on a side opposite to the first isolator 221 with respect to the capture region 211, and a first isolator 224 provided on a side opposite to the first isolator 222 with respect to the capture region 213, in addition to the first isolators 221 and 222, the first isolator 223 and the first isolator 224 being provided in the first direction d1. In addition, as illustrated in FIG. 3, each of the first isolators 221 to 224 crosses the surface of the substrate body 200 along the first direction d1.

In addition, in the present embodiment, the plurality of first isolators 22 are formed of a stimulus-responsive material such as a stimulus-responsive polymer. Specifically, as illustrated in FIG. 3, thin films formed of a stimulus-responsive material such as a stimulus-responsive polymer are formed on the substrate body 200 as the plurality of first isolators 22. The stimulus-responsive material is, for example, a temperature-responsive material whose wettability changes in response to a temperature or a light-responsive material whose wettability changes in response to light. For example, the temperature-responsive material is any one of poly(N-isopropylacrylamide) (PNIPA), poly(2-alkyl-2-oxazoline), a polypeptide containing proline, and the like. In addition, for example, the light-responsive material is any one of diarylethene, an azobenzene-based compound, a spiropyran-based compound, and the like. In the present embodiment, the plurality of first isolators 22 are formed of a temperature-responsive material.

The temperature-responsive material forming the plurality of first isolators 22 is a material that becomes hydrophilic in a case where the temperature is equal to or lower than a phase transition temperature and becomes hydrophobic when the temperature is equal to or higher than the phase transition temperature. The phase transition temperature is, for example, 32° C.

The flow path former 23 is a section provided on both sides of the plurality of capture regions 21 along the second direction d2 intersecting with the first direction d1. As illustrated in FIG. 3, the flow path former 23 according to the present embodiment is a region surrounded by an alternate long and short dash line on the substrate body 200, and is provided with a first flow path former 231 provided on a left side of each of the capture region 211, the capture region 212, and the capture region 213, and a second flow path formers 232 provided on a right side of each of the capture region 211, the capture region 212, and the capture region 213 along the second direction d2 orthogonal to the first direction d1. As illustrated in FIG. 3, each of the first flow path former 231 and the second flow path former 232 is provided so as to transverse the surface of the substrate body 200 along the second direction d2.

In addition, similarly to the plurality of first isolators 22, the flow path former 23 is a section whose wettability changes in response to a stimulus. In the present embodiment, the flow path former 23 is formed of a stimulus-responsive material such as a stimulus-responsive polymer. Specifically, as illustrated in FIG. 3, the flow path former 23 is provided by forming a thin film formed of a stimulus-responsive material such as a stimulus-responsive polymer on the substrate body 200 along the second direction d2, similarly to the plurality of first isolators 22. Since a specific example of the stimulus-responsive material is similar to the case of the plurality of first isolators 22 described above, the description thereof is omitted. In the present embodiment, the flow path former 23 is formed of a temperature-responsive material similarly to the plurality of first isolators 22.

The substrate body 200 is plate-like member. The substrate body 200 according to the present embodiment is formed of glass.

Note that, in the example illustrated in FIG. 3, the broken line and the alternate long and short dash line are shifted from the solid line so that the broken line indicating the plurality of first isolators 22, the alternate long and short dash line indicating the flow path former 23, and the solid line indicating the substrate body 200 and the plurality of capture regions 21 do not overlap each other.

Returning to FIG. 2, the configuration of the analysis apparatus 3 will be described. The analysis apparatus 3 is an apparatus that performs analysis on fine particles captured by the analysis substrate 2. As illustrated in FIG. 1, the analysis apparatus 3 includes an imager31, a stimulus applier 32, an input interface 33, an output interface 34, a storage circuitry 35, a communication interface 36, and a processing circuitry 37.

The imager 31 images the plurality of capture regions 21. Specifically, the imager 31 images the plurality of capture regions 21 of the analysis substrate 2 arranged in the analysis apparatus 3 under control of the processing circuitry 37. The imager 31 images the plurality of capture regions 21 of the analysis substrate 2 to detect a fluorescence signal of the EVs captured by each of the plurality of capture regions 21. The imager 31 is, for example, a camera including an imaging element. As the imaging element, a complementary metal oxide semiconductor (CMOS) or a charge coupled device (CCD) can be used, but the imaging element is not particularly limited thereto. In the present embodiment, the imager 31 is arranged above the analysis substrate 2, and images the plurality of capture regions 21 of the analysis substrate 2 by imaging from above. Note that the imager 31 may be arranged below the analysis substrate 2, and may image the plurality of capture regions 21 of the analysis substrate 2 by imaging from below.

The stimulus applier 32 applies a stimulus to the plurality of first isolators 22 of the analysis substrate 2. Specifically, the stimulus applier 32 applies a stimulus to the plurality of first isolators 22 of the analysis substrate 2 under control of the processing circuitry 37. In addition, the stimulus applier 32 according to the present embodiment applies a stimulus to the flow path former 23 under the control of the processing circuitry 37. In a case where the plurality of first isolators 22 of the analysis substrate 2 are formed of a temperature-responsive material, the stimulus applier 32 includes a Peltier element, an infrared irradiation mechanism that emits infrared rays, and other heat sources in order to apply heat to the temperature-responsive material. On the other hand, in a case where the plurality of first isolators 22 of the analysis substrate 2 are formed of a light-responsive material, the stimulus applier 32 includes a light source or the like in order to apply light to the light-responsive material. Since the plurality of first isolators 22 and the flow path former 23 according to the present embodiment are formed of a temperature-responsive material, the stimulus applier 32 according to the present embodiment includes, for example, an infrared irradiation mechanism.

The input interface 33 receives various input operations from a user, converts the received input operations into electrical signals, and outputs the electrical signals to the processing circuitry 37. The input interface 33 is connected to one or more input devices such as a membrane switch, a touch panel, a touch pad, a switch, a button, a joystick, and a trackball to which an instruction is input by touching an operation surface.

The output interface 34 is connected to the processing circuitry 37 and outputs a signal supplied from the processing circuitry 37. The output interface 34 is realized by, for example, a display circuitry, a print circuitry, an audio device, or the like. Examples of the display circuitry include a CRT display, a liquid crystal display, an organic EL display, an LED display, and a plasma display. Note that the display circuitry also includes a processing circuitry that converts data representing a display target into a video signal and outputs the video signal to the outside. The print circuitry includes, for example, a printer. Note that an output circuitry that outputs data representing a print target to the outside is also included in the print circuitry. The audio device includes, for example, a speaker. Note that an output circuitry that outputs an audio signal to the outside is also included in the audio device.

The storage circuitry 35 has a readable recording medium in a processor, such as a magnetic or optical recording medium or a semiconductor memory. The storage circuitry 35 stores a program executed by the circuitry of the analysis apparatus 3 according to the present embodiment. Note that some or all of the programs and data in a storage medium of the storage circuitry 35 may be downloaded via an electronic network.

In addition, the storage circuitry 35 stores one or more operation programs and the like according to the present embodiment. The operation program includes, for example, a program that defines a timing for executing predetermined processing necessary for measurement. The timing of executing the predetermined processing necessary for measurement is, for example, a timing of starting and stopping application of a stimulus by the stimulus applier 32, an imaging timing, an analysis timing, or the like. These timings are obtained empirically and experimentally in advance.

The communication interface 36 communicates with other devices under the control of the processing circuitry 37. Specifically, the communication interface 36 transmits an analysis result of the analysis substrate 2 to another device. For example, the communication interface 36 is realized by a network card, a network adapter, a network interface controller (NIC), or the like.

The processing circuitry 37 is a processor that functions as a center of the analysis apparatus 3. The processing circuitry 37 realizes a function corresponding to the control program by executing the operation program stored in the storage circuitry 35. Note that the processing circuitry 37 may include a storage area that stores at least part of the data stored in the storage circuitry 35.

The processing circuitry 37 illustrated in FIG. 2 realizes a function corresponding to the program by executing the operation program stored in the storage circuitry 35. For example, the processing circuitry 37 has a control function 371 and an analysis function 372 by executing an operation program. Note that, in the present embodiment, a case where the control function 371 and the analysis function 372 are realized by a single processor will be described, but the present invention is not limited thereto. For example, a processing circuitry may be configured by combining a plurality of independent processors, and each processor may execute a control program to realize these various functions.

The control function 371 is a function of integrally controlling each unit in the analysis apparatus 3 based on input information input from the input interface 33. The analysis function 372 analyzes an image captured by the imager 31. As a result, the presence or absence of EV expression and the number of EV expressions can be analyzed. Note that the control function 371 and the analysis function 372 illustrated in FIG. 2 constitute a controller and an analyzer, respectively, in the present embodiment.

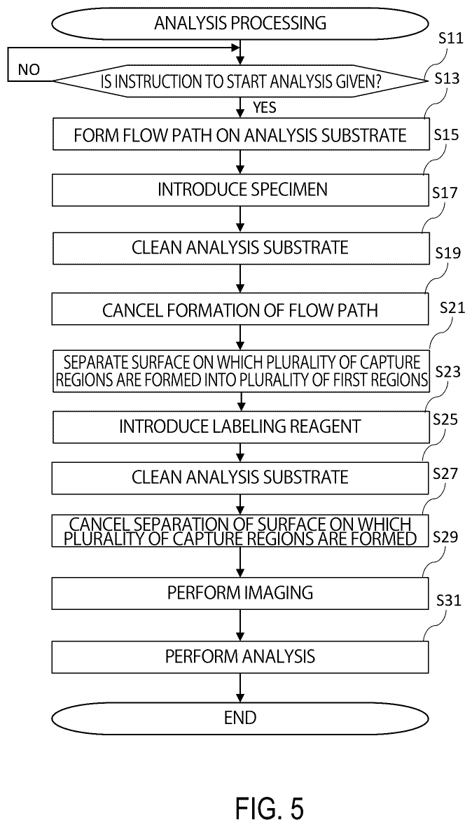

FIG. 5 is a flowchart for explaining contents of analysis processing performed by the analysis apparatus 3 according to the present embodiment. In this analysis processing, using the analysis substrate 2, a stimulus is applied to the flow path former 23 of the analysis substrate 2 to introduce a specimen into the plurality of capture regions 21, a stimulus is applied to the plurality of first isolators 22 to introduce a labeling reagent, a plurality of capture regions 21 of the analysis substrate 2 are imaged, or imaged images are analyzed. This analysis processing is processing executed in a case where an instruction to start analysis is given by a user. In addition, this analysis processing is, for example, processing executed at room temperature of 25° C., which is equal to or lower than a phase transition temperature of a temperature-responsive material.

As illustrated in FIG. 5, first, the analysis apparatus 3 determines whether or not an instruction to start analysis is given by a user (Step S11). The processing of determining whether or not an instruction to start analysis is given by a user is realized by the control function 371 in the processing circuitry 37. Specifically, after the analysis substrate 2 is arranged on the analysis apparatus 3 by the user, the analysis apparatus 3 determines whether or not the user gives an instruction to start analysis via the input interface 33. Then, in a case where the user does not give an instruction to start analysis (Step S11: No), the processing waits until the user gives an instruction to start analysis.

On the other hand, in a case where an instruction to start analysis is given by the user (Step S11: Yes), the analysis apparatus 3 forms a flow path on the analysis substrate 2 (Step S13). The processing of forming a flow path on the analysis substrate 2 is realized by the control function 371 in the processing circuitry 37. Specifically, the analysis apparatus 3 controls the infrared irradiation mechanism, which is the stimulus applier 32, to apply heat to the temperature-responsive material that forms the flow path former 23, such that wettability of the flow path former 23 changes, and a flow path for introducing a specimen into the surface of the analysis substrate 2 on which the plurality of capture regions 21 are formed is formed.

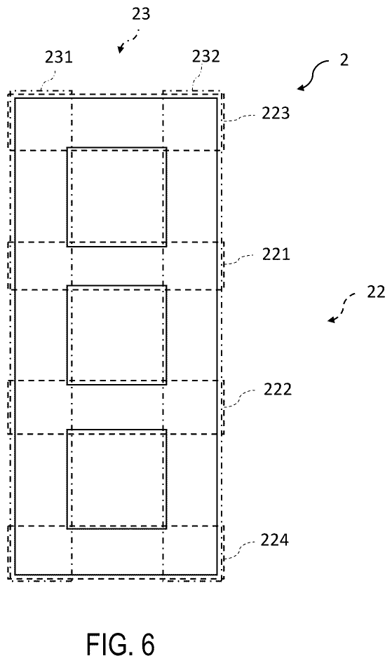

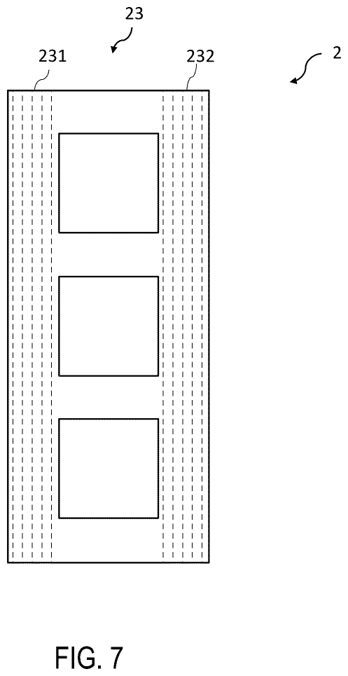

The formation of the flow path of the analysis substrate 2 according to the present embodiment will be described in detail with reference to FIGS. 6 and 7. FIG. 6 is a view illustrating an example of the analysis substrate 2 arranged in the analysis apparatus 3 in the first embodiment. FIG. 7 is a view illustrating an example of the analysis substrate 2 in which a stimulus is applied to the flow path former 23 by the analysis apparatus 3 according to the first embodiment. As illustrated in FIG. 6, in an initial state where no stimulus is applied to the flow path former 23, the wettability of the flow path former 23 of the analysis substrate 2 is, for example, hydrophilic. Here, the initial state refers to a state where the analysis substrate 2 is placed at a phase transition temperature or lower. Then, in Step S13, the analysis apparatus 3 controls the infrared irradiation mechanism as the stimulus applier 32 to irradiate the flow path former 23 with near infrared rays from above the analysis substrate 2 and apply heat to the flow path former 23.

When a temperature of the flow path former 23 is equal to or higher than the phase transition temperature, the wettability of the flow path former 23 of the substrate 2 or analysis changes. In the present embodiment, when the temperature of the flow path former 23 is equal to or higher than the phase transition temperature, the wettability of the flow path former 23 of the analysis substrate 2 becomes hydrophobic. In the example illustrated in FIG. 7, the change in wettability of the flow path former 23 to hydrophobic is indicated by hatching. Then, as illustrated in FIG. 7, the wettability of the flow path former 23 changes, such that a flow path for introducing a specimen is formed on the surface of the analysis substrate 2 on which the plurality of capture regions 21 are formed. Note that a heat dissipation mechanism may be arranged at positions corresponding to the plurality of capture regions 21 and the plurality of first isolators 22 on a back surface of the analysis substrate 2 so that the wettability of the temperature-responsive material forming the plurality of first isolators 22 does not change while heat is applied to the flow path former 23. Here, the back surface of the analysis substrate 2 is a surface placed on the analysis apparatus 3, and is a surface formed on a side opposite to the surface on which the plurality of first isolators 22 and the flow path former 23 are formed.

Next, as illustrated in FIG. 5, the analysis apparatus 3 introduces a specimen (Step S15). The processing of introducing the specimen is realized by the control function 371 in the processing circuitry 37. Specifically, the analysis apparatus 3 introduces a specimen containing EVs, which are fine particles, into the plurality of capture regions 21. More specifically, in a state where the wettability of the flow path former 23 is hydrophobic, the analysis apparatus 3 according to the present embodiment introduces EVs, which are fine particles, into the plurality of capture regions 21 by dropping the specimen onto the analysis substrate 2. Note that, in Step S15, the specimen may be introduced onto the analysis substrate 2 automatically or manually.

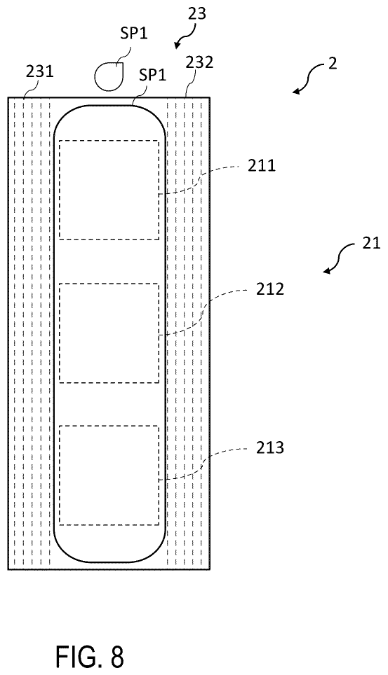

FIG. 8 is a view illustrating an example of the analysis substrate onto which a specimen SP1 is introduced in the first embodiment. As illustrated in FIG. 8, since the wettability of the flow path former 23 is hydrophobic in Step S13, in Step S15, the introduced specimen SP1 does not exceed the flow path former 23, and thus does not fall from the left and right direction of the analysis substrate 2. As a result, the possibility that the analysis apparatus 3 is contaminated with the specimen SP1 can be reduced. In addition, since the wettability of the plurality of first isolators 22 does not change and is hydrophilic, the introduced specimen SP1 can move along the flow path former 23. As such, the analysis apparatus 3 introduces the specimen SP1 into the plurality of capture regions 21.

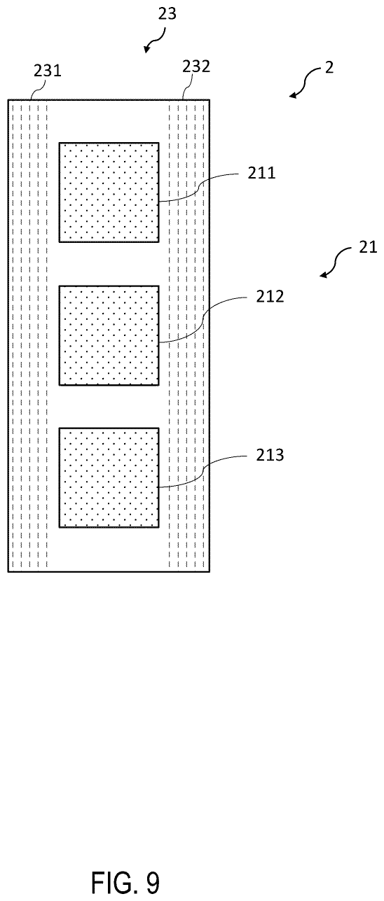

Next, as illustrated in FIG. 5, the analysis apparatus 3 cleans the analysis substrate 2 (Step S17). The processing of cleaning the analysis substrate 2 is realized by the control function 371 in the processing circuitry 37. Specifically, the analysis apparatus 3 removes the specimen SP1 introduced onto the analysis substrate 2, and cleans the analysis substrate 2 with a buffer solution (buffer) or the like. As a result, only the EVs captured by the plurality of capture regions 21 remain on the analysis substrate 2, and the EVs not captured by the plurality of capture regions 21 can be removed. Note that, in Step S17, the analysis substrate 2 may be cleaned automatically or manually. FIG. 9 is a view illustrating an example of the analysis substrate 2 on which the specimen is cleaned in the first embodiment. As illustrated in FIG. 9, the analysis substrate 2 is cleaned, such that the EVs not captured by the plurality of capture regions 21 are removed, and only the EVs captured by the capture regions 211 to 213 of the plurality of capture regions 21 remain on the analysis substrate 2.

Next, as illustrated in FIG. 5, the analysis apparatus 3 cancels the formation of the flow path (Step S19). The processing of canceling the formation of the flow path is realized by the control function 371 in the processing circuitry 37. Specifically, the analysis apparatus 3 controls the infrared irradiation mechanism, which is the stimulus applier 32, to stop applying heat to the temperature-responsive material forming the flow path former 23 so that the temperature of temperature-responsive material forming the flow path former 23 is equal to or lower than the phase transition temperature, such that the wettability of the flow path former 23 returns to hydrophilicity. As a result, the formation of the flow path is canceled. Note that the analysis apparatus 3 may include a cooling mechanism such as a fan to lower the temperature of the temperature-responsive material.

Next, as illustrated in FIG. 5, the analysis apparatus 3 separates the surface on which the plurality of capture regions 21 are formed into a plurality of first regions (Step S21). The process of separating the surface on which the plurality of capture regions 21 are formed into the plurality of first regions is realized by the control function 371 in the processing circuitry 37. Specifically, the analysis apparatus 3 separates the surface of the analysis substrate 2 on which the plurality of capture regions 21 are formed into a plurality of first regions each including at least one capture region and provided along the first direction d1. More specifically, the analysis apparatus 3 according to the present embodiment controls the infrared irradiation mechanism, which is the stimulus applier 32, to apply heat to the temperature-responsive material forming the plurality of first isolators 22, such that the wettability of the plurality of first isolators 22 changes, and the surface of analysis the substrate 2 on which the plurality of capture regions 21 are formed is separated into the plurality of first regions.

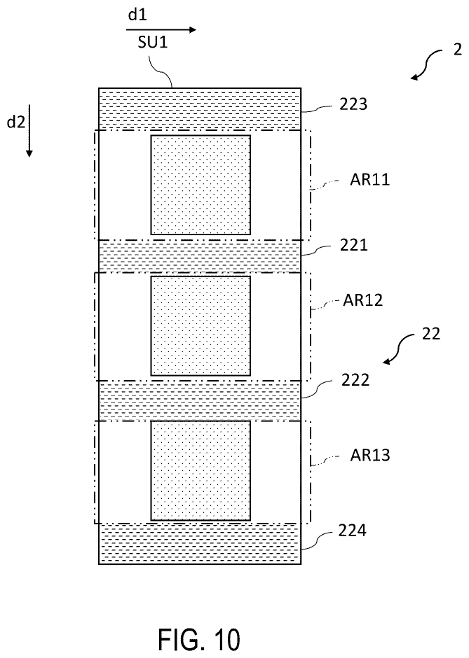

A method of separating the surface on which the plurality of capture regions 21 are formed into the plurality of first regions on the analysis substrate 2 according to the present embodiment will be described in detail with reference to FIGS. 6 and 10. FIG. 10 is a view illustrating an example of the analysis substrate 2 in which a stimulus is applied to the plurality of first isolators 22 by the analysis apparatus 3 according to the first embodiment. As illustrated in FIG. 6, in an initial state where no stimulus is applied to the plurality of first isolators 22, the wettability of the plurality of first isolators 22 of the analysis substrate 2 is, for example, hydrophilic. Then, as illustrated in FIG. 10, in Step S21, the analysis apparatus 3 controls the infrared irradiation mechanism, which is the stimulus applier 32, to irradiate the plurality of first isolators 22 with near infrared rays from above the analysis substrate 2 and apply heat to the plurality of first isolators 22.

When the temperature of the plurality of first isolators 22 is equal to or higher than the phase transition temperature, the wettability of the plurality of first isolators 22 of the analysis substrate 2 changes. In the present embodiment, when the temperature of the plurality of first isolators 22 is equal to or higher than the phase transition temperature, the wettability of the plurality of first isolators 22 of the analysis substrate 2 becomes hydrophobic. In the example illustrated in FIG. 10, similarly to the example illustrated in FIG. 7, the change in wettability of the plurality of first isolators 22 to hydrophobic is indicated by hatching. Then, as illustrated in FIG. 10, in Step S21, the wettability of the plurality of first isolators 22 of the analysis substrate 2 becomes hydrophobic, such that a surface SU1 of the analysis substrate 2 on which the plurality of capture regions 21 are formed is separated into a plurality of first regions AR11 to AR13 each including at least one capture region and provided along the first direction d1. As illustrated in FIG. 10, the first region AR11 includes the capture region 211, the first region AR12 includes the capture region 212, and the first region AR13 includes the capture region 213. Note that a heat dissipation mechanism may be arranged at positions corresponding to the plurality of capture regions 21 and the flow path former 23 on the back surface of the analysis substrate 2 so that the wettability of the temperature-responsive material forming the flow path former 23 does not change while heat is applied to the plurality of first isolators 22.

Next, as illustrated in FIG. 5, the analysis apparatus 3 introduces a labeling reagent (Step S23). The processing of introducing the labeling reagent is realized by the control function 371 in the processing circuitry 37. Specifically, the analysis apparatus 3 introduces labeling reagents different for each of the plurality of first regions AR11 to AR13 in a state where the wettability of the plurality of first isolators 22 is hydrophobic, that is, in a state where the isolators are separated into the plurality of first regions AR11 to AR13. Note that, in Step S23, the labeling reagent may be introduced onto the analysis substrate 2 automatically or manually.

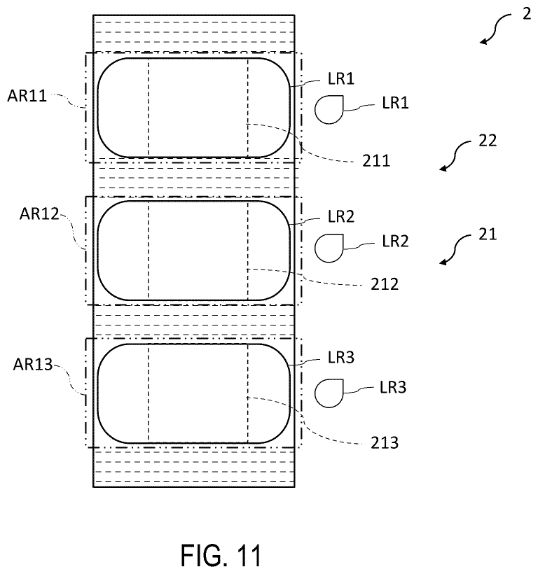

FIG. 11 is a view illustrating an example of the analysis substrate 2 onto which a labeling reagent is introduced in the first embodiment. As illustrated in FIG. 11, The labeling reagents LR introduced into each of the plurality of capture regions 21 are different, and Different labeling reagents LR1 to LR3 are introduced into each of the plurality of first regions AR11 to AR13. In the present embodiment, each of the labeling reagents LR1 to LR3 different for each of the plurality of first regions AR11 to AR13 contains a combination of different antibodies. In addition, the labeling reagents LR1 to LR3 different for each of the plurality of first regions AR11 to AR13 label EVs, which are fine particles, with fluorescent dyes different for each of the antibodies contained in the labeling reagents LR1 to LR3.

For example, the labeling reagent LR1 introduced into the capture region 211 included in the first region AR11 contains an antibody that binds to a biomarker (hereinafter, also referred to as BM) 1, an antibody that binds to BM2, an antibody that binds to BM3, and an antibody that binds to BM4 on the EVs captured in the capture region 211, and biomarkers on the EVs are labeled with fluorescent dyes different for each of the antibodies contained in the labeling reagent LR1. For example, the labeling reagent LR2 introduced into the capture region 212 included in the first region AR12 contains an antibody that binds to BM5, an antibody that binds to BM6, an antibody that binds to BM7, and an antibody that binds to BM8 on the EV captured in the capture region 212, and biomarkers on the EVs are labeled with fluorescent dyes different for each of the antibodies contained in the labeling reagent LR2. Furthermore, the labeling reagent LR3 introduced into the capture region 213 included in the first region AR13 contains an antibody that binds to BM9, an antibody that binds to BM10, an antibody that binds to BM11, and an antibody that binds to BM12 on the EVs captured in the capture region 213, and biomarkers on the EVs are labeled with fluorescent dyes different for each of the antibodies contained in the labeling reagent LR3. That is, the EVs, which are fine particles captured by each of the plurality of capture regions 21, are subjected to multiple stain with a plurality of fluorescent dyes.

In addition, as illustrated in FIG. 11, in Step S21, the wettability of the plurality of first isolators 22 is hydrophobic. Therefore, in Step S23, the labeling reagent LR1 introduced into the capture region 211 does not fall from a direction perpendicular to an introduction direction of the labeling reagent LR of the analysis substrate 2. In addition, the labeling reagent LR1 moves along the plurality of first isolators 22 without moving to the capture region 212 or the capture region 213. Similarly, the labeling reagent LR2 introduced into the capture region 212 moves along the plurality of first isolators 22 without moving to the capture region 211 or the capture region 213. Furthermore, the labeling reagent LR3 introduced into the capture region 213 does not fall from the direction perpendicular to the introduction direction of the labeling reagent LR of the analysis substrate 2. In addition, the labeling reagent LR3 moves along the plurality of first isolators 22 without moving to the capture region 211 or the capture region 212. That is, it is possible to introduce the labeling reagents LR1 to LR3 different for each of the plurality of first regions AR11 to AR13 while reducing a risk of mixing the labeling reagents LR1 to LR3.

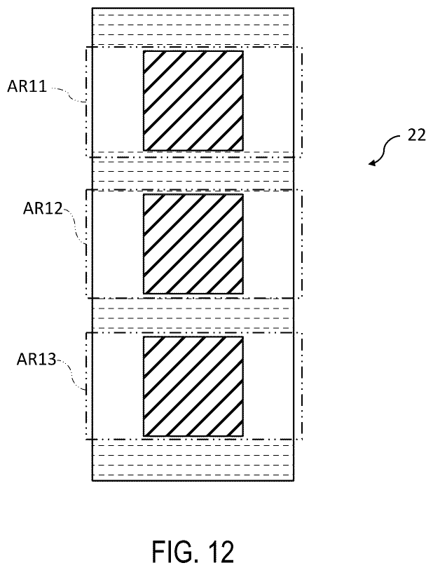

Next, as illustrated in FIG. 5, the analysis apparatus 3 cleans the analysis substrate 2 (Step S25). The processing of cleaning the analysis substrate 2 is realized by the control function 371 in the processing circuitry 37. Specifically, the analysis apparatus 3 removes the labeling reagents LR1 to LR3 introduced onto the analysis substrate 2, and cleans the analysis substrate 2 with pure water, a buffer, or the like. Note that, in Step S25, the analysis substrate 2 may be cleaned automatically or manually. FIG. 12 is a view illustrating an example of the analysis substrate 2 onto which cleaning is performed in the first embodiment. As illustrated in FIG. 12, the analysis substrate 2 is cleaned in a state where the wettability of the plurality of first isolators 22 is hydrophobic, that is, in a state where the isolators are separated into the plurality of first regions AR11 to AR13. As a result, when the analysis substrate 2 is cleaned, the risk of mixing the labeling reagents LR1 to LR3 can be reduced. Then, in each of the capture regions 211 to 213 of the plurality of capture regions 21, a combination of the different specimens SP and labeling reagents LR is hold.

Next, as illustrated in FIG. 5, the analysis apparatus 3 cancels the separation of the surface on which the plurality of capture regions 21 are formed (Step S27). The process of canceling the separation of the surface on which the plurality of capture regions 21 are formed is realized by the control function 371 in the processing circuitry 37. Specifically, the analysis apparatus 3 controls the infrared irradiation mechanism, which is the stimulus applier 32, to stop applying heat to the plurality of first isolators 22 so that the temperature of temperature-responsive material forming the plurality of first isolators 22 is equal to or lower than the phase transition temperature, such that the wettability of the plurality of first isolators 22 returns to hydrophilicity. As a result, the separation of the surface SU1 of the analysis substrate 2 on which the plurality of capture regions 21 are formed is canceled. Note that the analysis apparatus 3 may include a cooling mechanism such as a fan to lower the temperature of the temperature-responsive material of the plurality of first isolators 22.

Next, as illustrated in FIG. 5, the analysis apparatus 3 performs imaging (Step S29). The processing of imaging is realized by the control function 371 in the processing circuitry 37. Specifically, the analysis apparatus 3 controls the imager 31 to image the plurality of capture regions 21 of the analysis substrate 2. More specifically, the imager 31 images the plurality of capture regions 21 of the analysis substrate 2 to detect a fluorescence signal of the biomarkers on the EVs captured by each of the plurality of capture regions 21.

Next, as illustrated in FIG. 5, the analysis apparatus 3 performs analysis (Step S31). The processing of performing analysis is realized by the analysis function 372 in the processing circuitry 37. Specifically, the analysis apparatus 3 analyzes the image imaged in Step S29, and analyzes the presence or absence of EV expression and the number of EV expressions.

In Step S31, by analyzing the imaged image, the analysis processing according to the present embodiment is ended.

As described above, since the analysis substrate 2 according to the present embodiment includes the plurality of capture regions 21 that capture EVs, and the plurality of first isolators 22 whose wettability changes in response to a stimulus, the plurality of first isolators 22 being provided along the first direction d1 between the capture regions 21, the surface SU1 of the analysis substrate 2 on which the plurality of capture regions 21 are formed can be separated into the plurality of first regions AR11 to AR13 each including at least one capture region and provided along the first direction d1, and the labeling reagent introduced into each of the plurality of first regions AR11 to AR13 can be prevented from mixing.

That is, on one analysis substrate 2, a plurality of biomarkers in one specimen can be simultaneously analyzed, such that the analysis efficiency can be improved, and a throughput of the fine particle analysis can be improved.

### Second Embodiment

In the analysis system 1 according to the first embodiment described above, the plurality of biomarkers in one specimen can be simultaneously analyzed on the analysis substrate 2, but the plurality of biomarkers may be simultaneously analyzed for each of the plurality of specimens in one analysis substrate. In the second embodiment, an analysis substrate capable of introducing a plurality of specimens and simultaneously analyzing a plurality of biomarkers for each of the plurality of specimens will be described. Hereinafter, parts different from the first embodiment described above will be described.

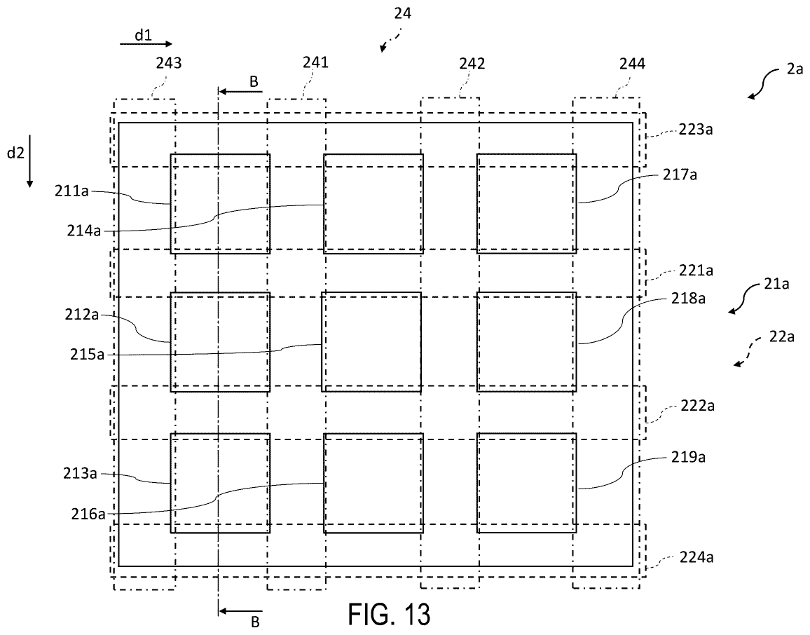

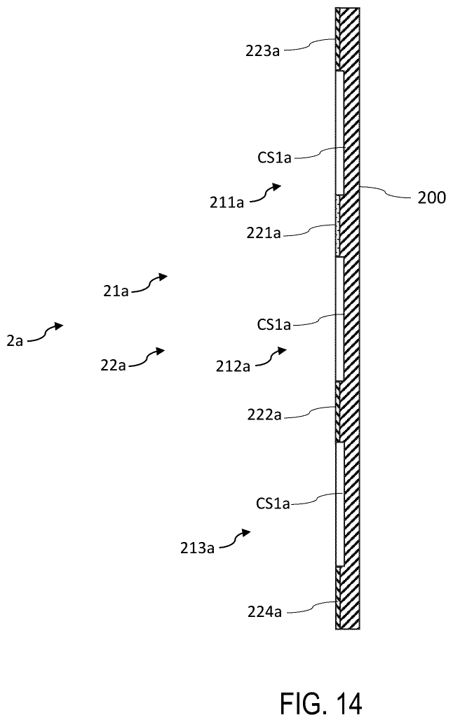

FIG. 13 is a top view illustrating an example of an analysis substrate 2a according to the second embodiment, and is a view corresponding to FIG. 3 in the first embodiment described above. FIG. 14 is a cross-sectional view taken along line B-B in FIG. 13, and is a view corresponding to FIG. 4 in the first embodiment described above. The analysis substrate 2a according to the present embodiment is a gold substrate. In addition, as illustrated in FIGS. 13 and 14, the analysis substrate 2a according to the present embodiment includes a plurality of capture regions 21a, a plurality of first isolators 22a, a plurality of second isolators 24, and the substrate body 200. Note that, since a specific configuration of the substrate body 200 is the same as that in the first embodiment described above, the description thereof is omitted.

As the plurality of capture regions 21a according to the present embodiment, nine capture regions 211a to 219a are provided. As illustrated in FIG. 13, the nine capture regions 211a to 219a are provided at a predetermined interval along a first direction d1 and a second direction d2. Surfaces of the plurality of capture regions 21a according to the present embodiment are surfaces capable of physically adsorbing to fine particles. Specifically, a capture face CS1a, which is the surface of each of the capture regions 211a to 219a illustrated in FIG. 14, is a surface treated with citric acid. Note that, in the example illustrated in FIG. 13, nine capture regions are provided, but the number of the plurality of capture regions 21a provided on the substrate body 200 is not limited to nine. That is, the number of the plurality of capture regions 21a is arbitrary, and may be four or more and eight or less, or may be ten or more. Since the other configurations of the plurality of capture regions 21a are similar to those of the plurality of capture regions 21 according to the first embodiment described above, the description thereof is omitted.

As illustrated in FIG. 13, each of the plurality of first isolators 22a according to the present embodiment is a region surrounded by the broken line on the substrate body 200, and includes a first isolator 221a provided along the first direction d1 between the capture region 211a and the capture region 212a, between the capture region 214a and the capture region 215a, and between the capture region 217a and the capture region 218a, and a first isolator 222a provided along the first direction d1 between the capture region 212a and the capture region 213a, between the capture region 215a and the capture region 216a, and between the capture region 218a and the capture region 219a. In addition, each of the plurality of first isolators 22a according to the present embodiment includes a first isolator 223a provided on a side opposite to the capture region 211a and the capture region 212a with respect to the capture region 211a, and a first isolator 224a provided on a side opposite to the capture region 212a and the capture region 213a with respect to the capture region 213a, in addition to the first isolators 221a and 222a, the first isolator 223a and the first isolator 224a being provided along the second direction d2. In addition, as illustrated in FIG. 13, each of the first isolators 221a to 224a crosses the surface of the substrate body 200 along the first direction d1. Since the other configurations of the plurality of first isolators 22a are similar to those of the plurality of first isolators 22 according to the first embodiment described above, the description thereof is omitted.

Each of the plurality of second isolators 24 is a section provided between the capture regions along the second direction d2 intersecting with the first direction d1. Each of the plurality of second isolators 24 according to the present embodiment is provided between the capture regions along the second direction d2 orthogonal to the first direction d1. As illustrated in FIG. 13, each of the plurality of second isolators 24 according to the present embodiment is a region surrounded by the broken line on the substrate body 200, and includes a second isolator 241 provided along the second direction d2 between the capture region 211a and the capture region 214a, between the capture region 212a and the capture region 215a, and between the capture region 213a and the capture region 216a, and a second isolator 242 provided along the second direction d2 between the capture region 214a and the capture region 217a, between the capture region 215a and the capture region 218a, and between the capture region 216a and the capture region 219a. In addition, the second isolator 24 according to the present embodiment includes a first isolator 223a on a side opposite to the second isolator 241 with respect to the capture region 211a and a first isolator 224a on a side opposite to the second isolator 242 with respect to the capture region 213a, the first isolator 223a and the first isolator 224a being provided along the second direction d2, in addition to the second isolators 241 and 242. In addition, as illustrated in FIG. 13, each of the second isolators 241 to 244 crosses the surface of the substrate body 200 along the second direction d2.

In addition, similarly to the plurality of first isolators 22a, each of the plurality of second isolators 24 according to the present embodiment is a section whose wettability changes in response to a stimulus. The plurality of second isolators 24 are formed of a stimulus-responsive material such as a stimulus-responsive polymer. In the present embodiment, as illustrated in FIG. 14, thin films formed of a stimulus-responsive material such as a stimulus-responsive polymer are formed on the substrate body 200 as the plurality of second isolators 24. Since a specific example of the stimulus-responsive material is similar to the case of the plurality of first isolators 22 in the first embodiment described above, the description thereof is omitted. In the present embodiment, the plurality of second isolators 24 are formed of a temperature-responsive material similarly to the plurality of first isolators 22a.

Note that, in the example illustrated in FIG. 13, the broken line and the alternate long and short dash line are shifted from the solid line so that the broken line indicating the plurality of first isolators 22a, the alternate long and short dash line indicating the plurality of second isolators 24, and the solid line indicating the substrate body 200 and the plurality of capture regions 21a do not overlap each other.

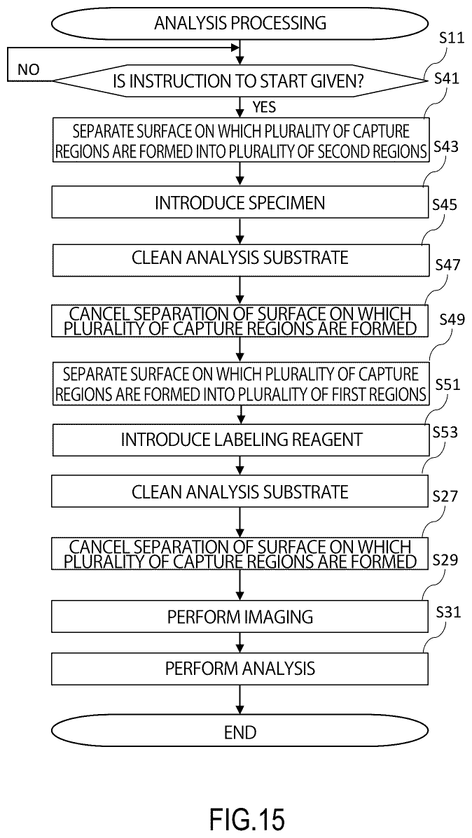

FIG. 15 is a flowchart for explaining contents of analysis processing performed by an analysis apparatus 3 according to the second embodiment. In this analysis processing, a stimulus is applied to the plurality of second isolators 24 of the analysis substrate 2a to introduce a specimen SP into the plurality of capture regions 21a, a stimulus is applied to the plurality of first isolators 22a to introduce a labeling reagent LR, a plurality of capture regions 21a of the analysis substrate 2a are imaged, or imaged images are analyzed. This analysis processing is processing executed in a case where an instruction to start analysis is given by a user. Note that the processing of Step S11 illustrated in FIG. 15 is equivalent to that in FIG. 5 in the first embodiment described above, and thus the description thereof is omitted.

On the other hand, in a case where an instruction to start analysis is given by the user (Step S11: Yes), the analysis apparatus 3 separates a surface on which the plurality of capture regions 21a are formed into a plurality of second regions (Step S41). The process of separating the surface on which the plurality of capture regions 21a are formed into the plurality of second regions is realized by a control function 371 in a processing circuitry 37. Specifically, the analysis apparatus 3 separates the surface of the analysis substrate 2a on which the plurality of capture regions 21a are formed into the plurality of second regions each including at least one capture region and provided along the second direction d2 intersecting with the first direction d1. More specifically, the analysis apparatus 3 according to the present embodiment controls an infrared irradiation mechanism, which is a stimulus applier 32, to apply heat to the temperature-responsive material forming the plurality of second isolators 24, such that the wettability of the plurality of second isolators 24 changes, and a surface SU1 of the analysis substrate 2a on which the plurality of capture regions 21a are formed is separated into the plurality of second regions.

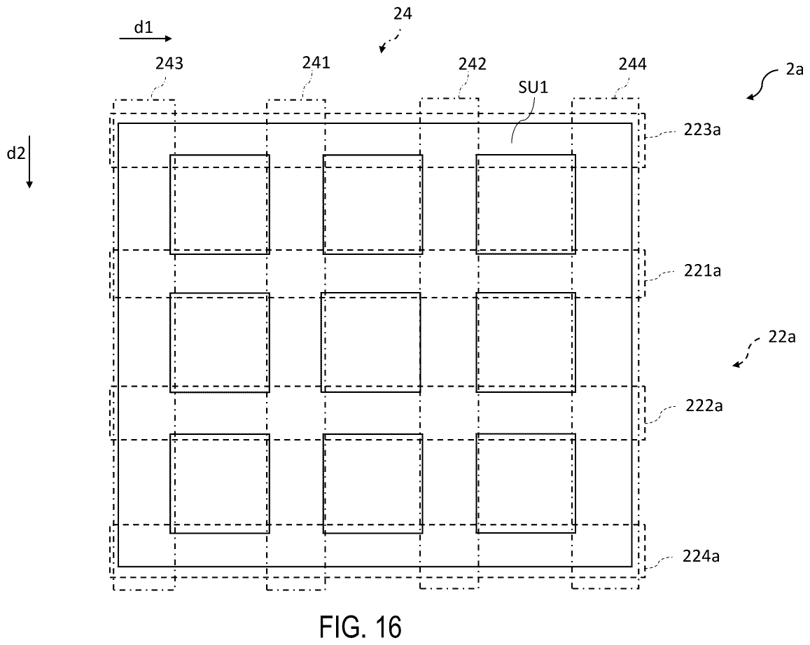

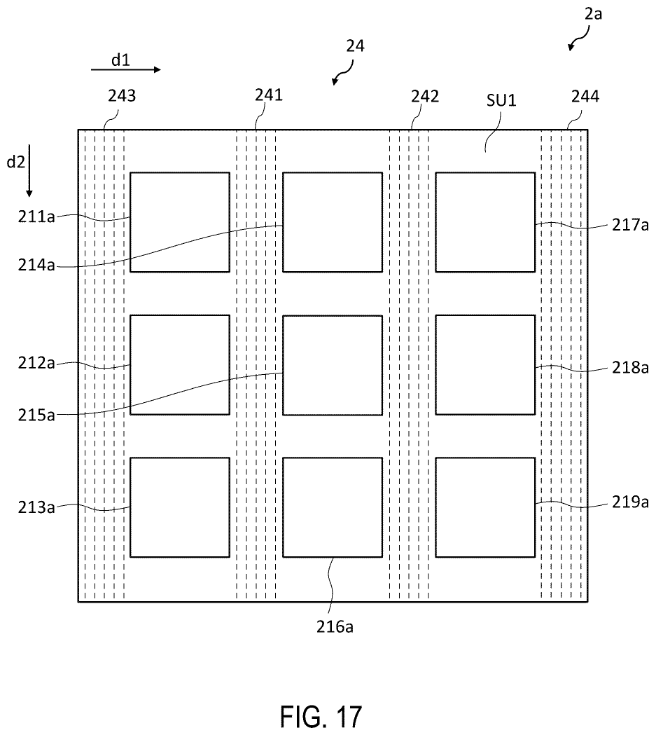

A method of separating the surface SU1 on which the plurality of capture regions 21 are formed into the plurality of second regions on the analysis substrate 2a according to the present embodiment will be described in detail with reference to FIGS. 16 and 17. FIG. 16 is a view illustrating an example of the analysis substrate 2a arranged in the analysis apparatus 3 according to the second embodiment, and is a view corresponding to FIG. 6 in the first embodiment described above. FIG. 17 is a view illustrating an example of the analysis substrate 2a in which a stimulus is applied to the plurality of second isolators 24 by the analysis apparatus 3 according to the second embodiment. As illustrated in FIG. 16, in an initial state where no stimulus is applied to the plurality of second isolators 24, the wettability of the plurality of second isolators 24 of the analysis substrate 2a is, for example, hydrophilic. Then, as illustrated in FIG. 16, in Step S41, the analysis apparatus 3 controls the infrared irradiation mechanism, which is the stimulus applier 32, to irradiate the plurality of second isolators 24 with near infrared rays from above the analysis substrate 2a and apply heat to the plurality of first isolators 22a.

When a temperature of the plurality of second isolators 24 is equal to or higher than a phase transition temperature, the wettability of the plurality of second isolators 24 of the analysis substrate 2a changes. In the present embodiment, when the temperature of the plurality of second isolators 24 is equal to or higher than the phase transition temperature, the wettability of the plurality of second isolators 24 of the analysis substrate 2a becomes hydrophobic. In the example illustrated in FIG. 16, the change in wettability of the plurality of second isolators 24 to hydrophobic is indicated by hatching. Then, as illustrated in FIG. 17, the wettability of the plurality of second isolators 24 of the analysis substrate 2a becomes hydrophobic, such that the surface SU1 of the analysis substrate 2a on which the plurality of capture regions 21 are formed is separated into a plurality of second regions AR21 to AR23 each including at least one capture region and provided along the second direction d2. As illustrated in FIG. 17, the second region AR21 includes the capture regions 211a to 213a, the second region AR22 includes the capture regions 214a to 216a, and the second region AR23 includes the capture regions 217a to 219a. Note that a heat dissipation mechanism may be arranged at positions corresponding to the plurality of capture regions 21 and the plurality of first isolators 22a on a back surface of the analysis substrate 2a so that the wettability of the temperature-responsive material forming the plurality of first isolators 22a does not change while heat is applied to the plurality of second isolators 24.

Next, as illustrated in FIG. 15, the analysis apparatus 3 introduces a specimen SP (Step S43). The processing of introducing the specimen SP is realized by the control function 371 in the processing circuitry 37. Specifically, the analysis apparatus 3 introduces the specimen SP into the plurality of capture regions 21a by introducing the specimens SP different for each of the plurality of second regions AR21 to AR23 in a state where the wettability of the plurality of second isolators 24 is hydrophobic, that is, in a state where the isolators are separated into the plurality of second regions AR21 to AR23. Note that, in Step S43, the specimen SP may be introduced onto the analysis substrate 2a automatically or manually.

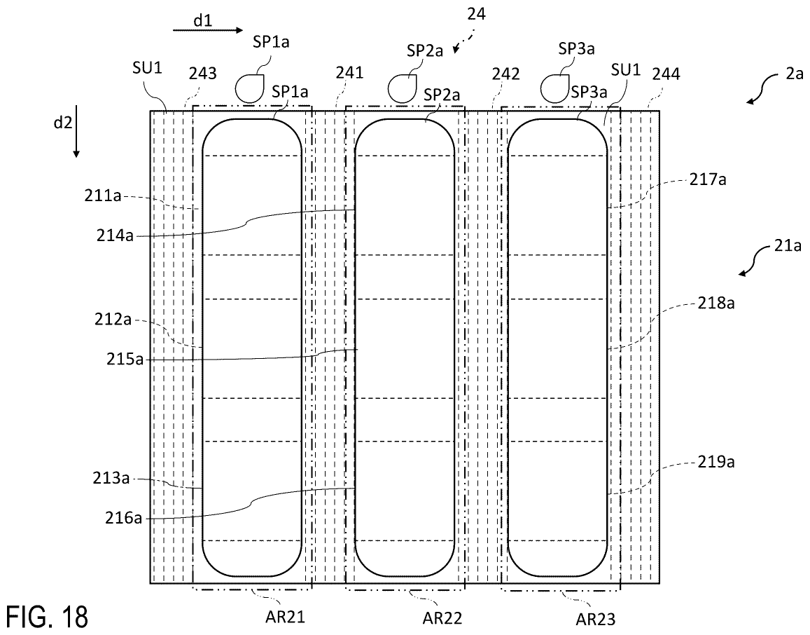

FIG. 18 is a view illustrating an example of the analysis substrate 2a onto which the specimen SP is introduced in the second embodiment, and is a view corresponding to FIG. 8 in the first embodiment described above. As illustrated in FIG. 18, in the present embodiment, specimens SP1a to SP3a different for each of the plurality of second regions AR21 to AR23 are introduced. In the present embodiment, the specimens SP1a to SP3a different for each of the plurality of second regions AR21 to AR23 are the same specimens and are specimens diluted to different concentrations.

For example, a specimen diluted to have a concentration of X % is introduced as the specimen SP1a into the capture regions 211a to 213a included in the second region AR21. In addition, a specimen diluted to have a concentration of Y % is introduced as the specimen SP2a into the capture regions 214a to 216a included in the second region AR22. Furthermore, a specimen diluted to have a concentration of Z % is introduced as the specimen SP3a into the capture regions 217a to 219a included in the second region AR23.

In addition, as illustrated in FIG. 18, in Step S41, the wettability of the plurality of second isolators 24 is hydrophobic. Therefore, in Step S43, the specimen SP1a introduced into the capture regions 211a to 213a included in the second region AR21 does not fall from a direction perpendicular to an introduction direction of the specimen SP1a of the analysis substrate 2a. In addition, the specimen SP1a moves along the second isolators 241 and 243 without moving to the second region AR22 or the second region AR23. Similarly, the specimen SP2a introduced into the capture regions 214a to 216a included in the second region AR22 moves along the second isolators 241 and 242 without moving to the second region AR21 or the second region AR23. Furthermore, the specimen SP3a introduced into the capture regions 217a to 219a included in the second region AR23 does not fall from a direction perpendicular to the introduction direction of the specimen SP3a of the analysis substrate 2a. In addition, the specimen SP3a moves along the second isolators 242 and 244 without moving to the second region AR21 or the second region AR22. That is, it is possible to introduce the specimens SP1a to SP3a different for each of the plurality of second regions AR21 to AR23 while reducing a risk of mixing the specimens SP1a to SP3a.

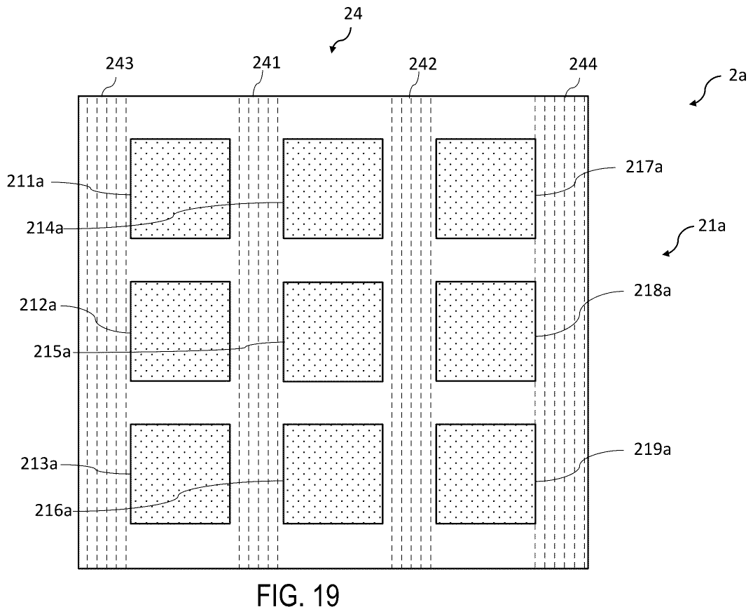

Next, as illustrated in FIG. 15, the analysis apparatus 3 cleans the analysis substrate 2a (Step S45). The processing of cleaning the analysis substrate 2a is realized by the control function 371 in the processing circuitry 37. Specifically, the analysis apparatus 3 removes the specimens SP1a to SP3a introduced onto the analysis substrate 2a, and cleans the analysis substrate 2a with pure water, a buffer, or the like. Note that, in Step S45, the analysis substrate 2a may be cleaned automatically or manually. FIG. 19 is a view illustrating an example of the analysis substrate 2a onto which the specimen is cleaned in the second embodiment, and is a view corresponding to FIG. 9 in the first embodiment described above. As illustrated in FIG. 19, the analysis substrate 2a is cleaned in a state where the wettability of the plurality of second isolators 24 is hydrophobic, such that the risk of mixing the specimens SP1a to SP3a can be reduced.

Next, as illustrated in FIG. 15, the analysis apparatus 3 cancels the separation of the surface SU1 on which the plurality of capture regions 21a are formed (Step S47). The process of canceling the separation of the surface SU1 on which the plurality of capture regions 21a are formed is realized by the control function 371 in the processing circuitry 37. Specifically, the analysis apparatus 3 controls the infrared irradiation mechanism, which is the stimulus applier 32, to stop applying heat to the plurality of second isolators 24 so that the temperature of temperature-responsive material forming the plurality of second isolators 24 is equal to or lower than the phase transition temperature, such that the wettability of the plurality of second isolators 24 returns to hydrophilicity. As a result, the separation of the surface SU1 of the analysis substrate 2a on which the plurality of capture regions 21a are formed is canceled. Note that the analysis apparatus 3 may include a cooling mechanism such as a fan to lower the temperature of the temperature-responsive material of the plurality of second isolators 24.

Next, as illustrated in FIG. 15, the analysis apparatus 3 separates the surface SU1 on which the plurality of capture regions 21a are formed into a plurality of first regions (Step S49). The process of separating the surface SU1 on which the plurality of capture regions 21a are formed into the plurality of first regions is realized by a control function 371 in a processing circuitry 37. Specifically, the analysis apparatus 3 separates the surface of the analysis substrate 2a on which the plurality of capture regions 21a are formed into a plurality of first regions each including at least one capture region and provided along the first direction d1. More specifically, the analysis apparatus 3 according to the present embodiment controls the infrared irradiation mechanism, which is the stimulus applier 32, to apply heat to the temperature-responsive material forming the plurality of first isolators 22a, such that the wettability of the plurality of first isolators 22a changes, and the surface of the analysis substrate 2a on which the plurality of capture regions 21a are formed is separated into the plurality of first regions.

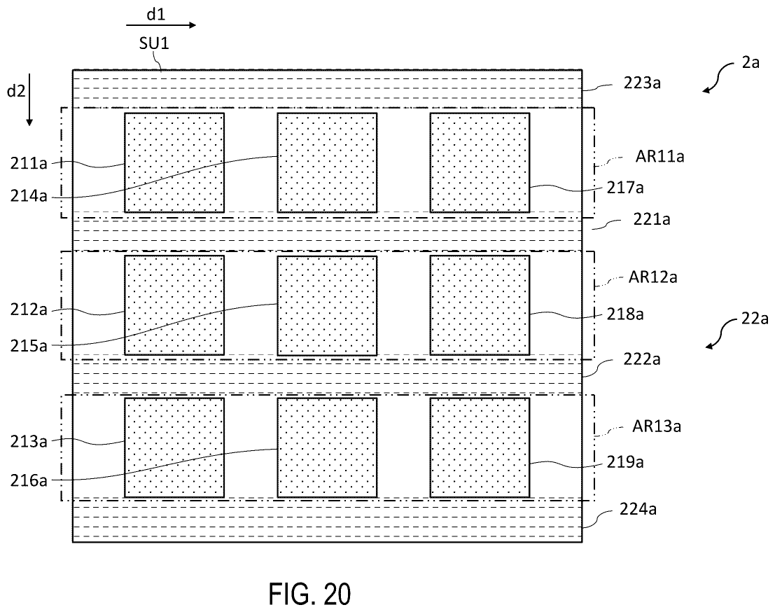

A method of separating the surface SU1 on which the plurality of capture regions 21a are formed into the plurality of first regions on the analysis substrate 2a according to the present embodiment will be described in detail with reference to FIGS. 16 and 20. FIG. 20 is a view illustrating an example of the analysis substrate 2a in which a stimulus is applied to the plurality of first isolators 22a by the analysis apparatus 3 according to the second embodiment, and is a view corresponding to FIG. 10 in the first embodiment described above. In the present embodiment, as illustrated in FIG. 20, in Step S49, the wettability of the plurality of first isolators 22a of the analysis substrate 2a becomes hydrophobic, such that the surface SU1 of the analysis substrate 2a on which the plurality of capture regions 21a are formed is separated into a plurality of first regions AR11a to AR13a each including at least one capture region and provided along the first direction d1. As illustrated in FIG. 20, the first region AR11a includes the capture regions 211a, 214a, and 217a, the first region AR12a includes the capture regions 212a, 215a, and 218a, and the first region AR13a includes the capture region 213a, 216a, and 219a. Since the description other than the method of separating the surface SU1 on which the plurality of capture regions 21a are formed into the plurality of first regions in FIGS. 16 and 20 described above is the same as the description of FIG. 10 in the first embodiment described above, the description is omitted.

Next, as illustrated in FIG. 15, the analysis apparatus 3 introduces a labeling reagent (Step S51). The processing of introducing the labeling reagent is realized by the control function 371 in the processing circuitry 37. Specifically, the analysis apparatus 3 introduces labeling reagents different for each of the plurality of first regions AR11a to AR13a in a state where the wettability of the plurality of first isolators 22a is hydrophobic, that is, in a state where the first isolators 22a are separated into the plurality of first regions AR11a to AR13a. Note that, in Step S51, the labeling reagent may be introduced onto the analysis substrate 2a automatically or manually.

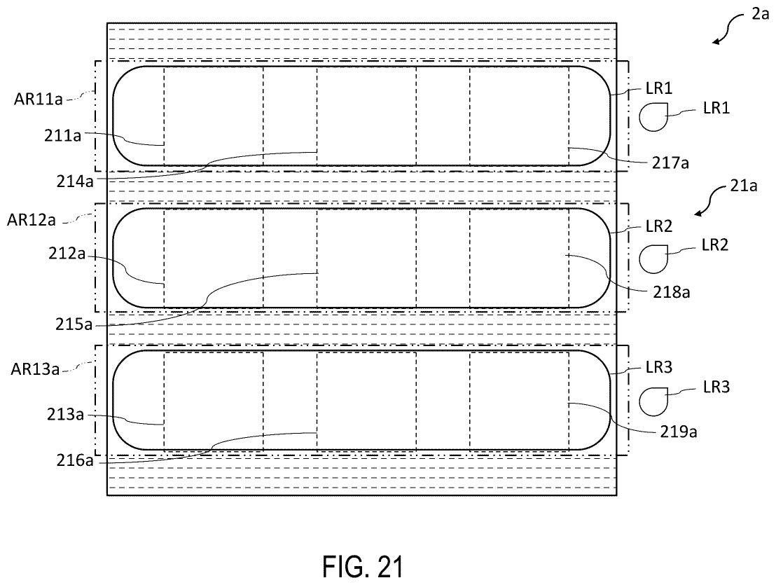

FIG. 21 is a view illustrating an example of the analysis substrate 2a onto which the labeling reagent LR is introduced in the second embodiment, and is a view corresponding to FIG. 11 in the first embodiment described above. For example, as illustrated in FIG. 21, the labeling reagent LR1 introduced into each of the capture regions 211a, 214a, and 217a included in the first region AR11a contains an antibody that binds to BM1, an antibody that binds to BM2, an antibody that binds to BM3, and an antibody that binds to BM4 on the EVs captured in each of the capture regions 211a, 214a, and 217a, and biomarkers on the EVs are labeled with fluorescent dyes different for each of antibodies contained in the labeling reagent LR1. The labeling reagent LR2 introduced into each of the capture regions 212a, 215a, and 218a included in the first region AR12a contains an antibody that binds to BM5, an antibody that binds to BM6, an antibody that binds to BM7, and an antibody that binds to BM8 on the EVs captured in each of the capture regions 212a, 215a, and 218a, and biomarkers on the EVs are labeled with fluorescent dyes different for each of antibodies contained in the labeling reagent LR2. Furthermore, the labeling reagent LR3 introduced into each of the capture regions 213a, 216a, and 219a included in the first region AR13a contains an antibody that binds to BM9, an antibody that binds to BM10, an antibody that binds to BM11, and an antibody that binds to BM12 on the EVs captured in each of the capture regions 213a, 216a, and 219a, and biomarkers on the EVs are labeled with fluorescent dyes different for each of antibodies contained in the labeling reagent LR3.

In addition, as illustrated in FIG. 21, in Step S49, the wettability of the plurality of first isolators 22a is hydrophobic. Therefore, in Step S51, the labeling reagent LR1 introduced into each of the capture regions 211a, 214a, and 217a does not fall from a direction perpendicular to an introduction direction of the labeling reagent LR1 of the analysis substrate 2a. In addition, the labeling reagent LR1 moves along the first isolators 221a and 223a without moving to the first region AR12a or the first region AR13a. Similarly, the labeling reagent LR2 introduced into each of the capture regions 212a, 215a, and 218a moves along the first isolators 221a and 222a without moving to the first region AR11a or the first region AR13a. Furthermore, the labeling reagent LR3 introduced into each of the capture regions 213a, 216a, and 219a does not fall from the direction perpendicular to the introduction direction of the labeling reagent LR of the analysis substrate 2a. In addition, the labeling reagent LR3 moves along the first isolators 222a and 224a without moving to the first region AR11a or the first region AR12a. That is, it is possible to introduce the labeling reagents LR1 to LR3 different for each of the plurality of first regions AR11a to AR13a while reducing a risk of mixing the labeling reagents LR1 to LR3.

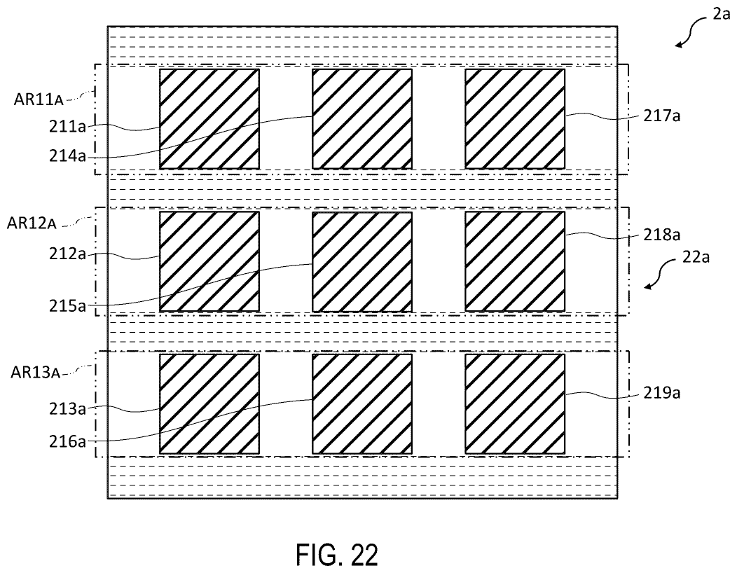

Next, as illustrated in FIG. 15, the analysis apparatus 3 cleans the analysis substrate 2a (Step S53). The processing of cleaning the analysis substrate 2a is realized by the control function 371 in the processing circuitry 37. Specifically, the analysis apparatus 3 removes the labeling reagents LR1 to LR3 introduced onto the analysis substrate 2a, and cleans the analysis substrate 2a with pure water, a buffer, or the like. Note that, in Step S53, the analysis substrate 2a may be cleaned automatically or manually. FIG. 22 is a view illustrating an example of the analysis substrate 2a onto which cleaning is performed in the second embodiment. As illustrated in FIG. 22, the analysis substrate 2a is cleaned in a state where the wettability of the plurality of first isolators 22a is hydrophobic, that is, in a state where the isolators are separated into the plurality of first regions AR11a to AR13a. As a result, when the analysis substrate 2a is cleaned, the risk of mixing the labeling reagents LR1 to LR3 can be reduced. Then, in each of the capture regions 211a to 219a of the plurality of capture regions 21a, a combination of the different specimens SP and labeling reagents LR is hold. Since the processing from Step S27 to Step S31 after Step S53 is the same as that in FIG. 5 in the first embodiment described above, the description thereof is omitted. Then, in Step S31, by analyzing the imaged image, the analysis processing according to the present embodiment is ended.

As described above, since the analysis substrate 2a according to the present embodiment includes the plurality of capture regions 21 that capture EVs, the plurality of first isolators 22a whose wettability changes in response to a stimulus, the plurality of first isolators 22a being provided along the first direction d1 between the capture regions, and the plurality of second isolators 24 provided along the second direction d2 between the capture regions, when the specimens SP1a to SP3a are introduced, the wettability of the plurality of second isolators 24 is changed by applying a stimulus to the plurality of second isolators 24, such that it is possible to prevent the different specimens from being mixed with each other, and when the labeling reagents LR1 to LR3 are introduced, the wettability of each of the plurality of first isolators 22a is changed by applying a stimulus to each of the plurality of first isolators 22a, such that it is possible to prevent the different labeling reagents from being mixed with each other. That is, on one analysis substrate 2a, a plurality of biomarkers in a plurality of specimens can be simultaneously analyzed, such that the analysis efficiency can be improved, and a throughput of the fine particle analysis can be improved.

### Third Embodiment

In the analysis systems 1 according to the first and second embodiments described above, the plurality of capture regions 21 and 21a of the analysis substrates 2 and 2a may be configured so that plasmon resonance occurs. Therefore, in a third embodiment, an analysis system capable of using an analysis substrate configured to generate plasmon resonance will be described. Hereinafter, parts different from the first and second embodiments described above will be described.

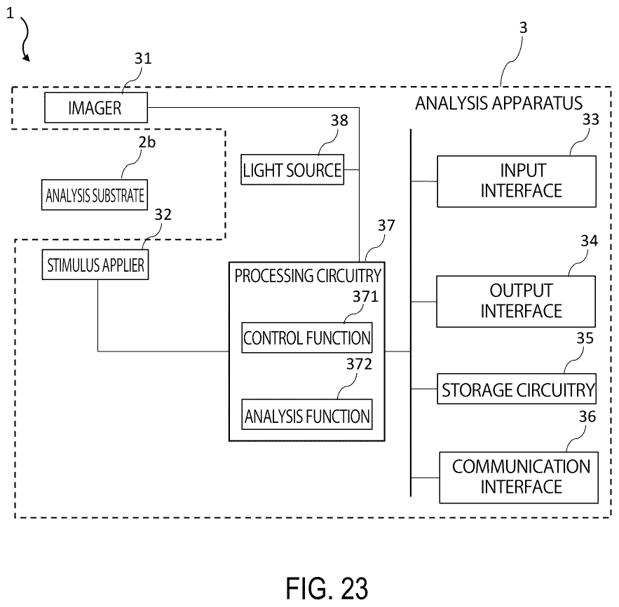

FIG. 23 is a block diagram illustrating an example of a configuration of an analysis system 1 according to the third embodiment, and is a view corresponding to FIG. 2 in the first embodiment described above. As illustrated in FIG. 23, since the configurations of the analysis substrate and the stimulus applier are different from those of the first embodiment, in the present embodiment, they are referred to as an analysis substrate 2b and a stimulus applier 32a. In addition, a light source 38 is added to the analysis system 1 according to the present embodiment. Note that, since configurations other than the analysis substrate 2b, the stimulus applier 32a, and the light source 38 are similar to those in FIG. 1 in the first embodiment described above, description thereof is omitted.

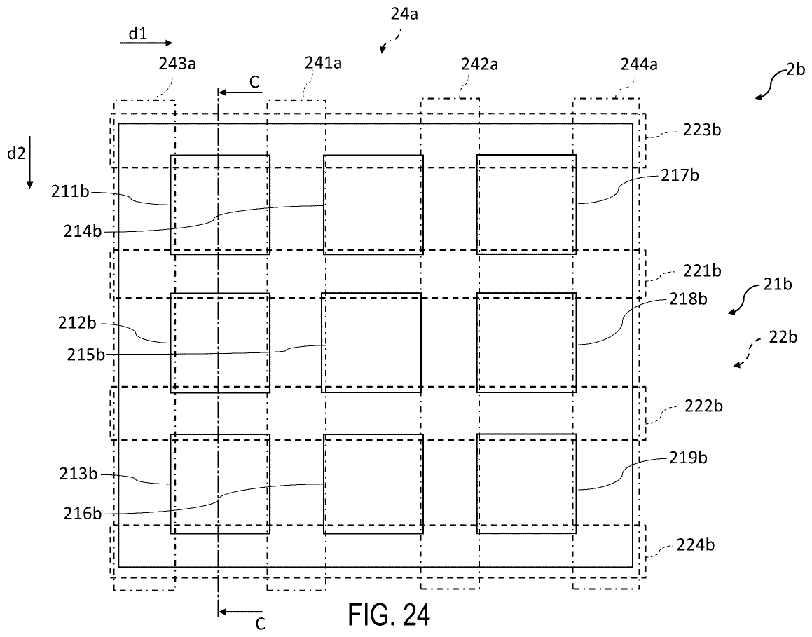

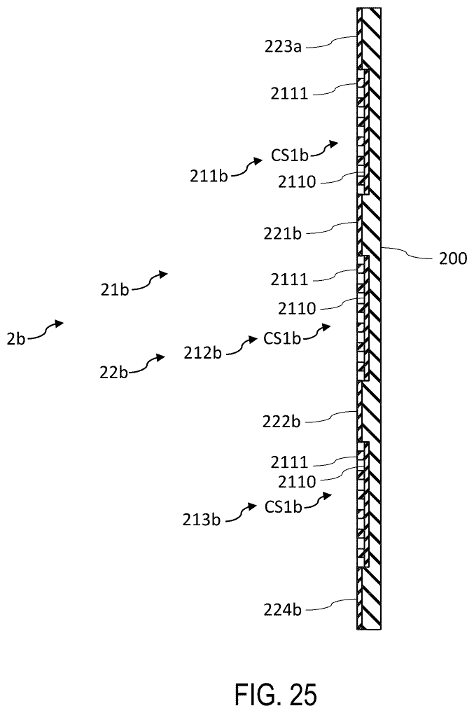

The analysis substrate 2b according to the third embodiment will be described with reference to FIGS. 24 and 25. FIG. 24 is a top view illustrating an example of the analysis substrate 2b according to the third embodiment, and is a view corresponding to FIG. 14 in the second embodiment described above. FIG. 25 is a cross-sectional view taken along ling C-C in FIG. 24. In addition, as illustrated in FIGS. 24 and 25, the analysis substrate 2b according to the present embodiment includes a plurality of capture regions 21b, a plurality of first isolators 22b, a plurality of second isolators 24a, and the substrate body 200.

Each of the plurality of capture regions 21b according to the present embodiment is a region where surface plasmon resonance occurs by irradiation with excitation light of the light source 38. Specifically, as illustrated in FIG. 25, the plurality of capture regions 21b according to the present embodiment include a metal film 2110 on which a diffraction grating 2111 is formed. The metal film 2110 is formed on the surfaces of the plurality of capture regions 21b by, for example, vapor deposition. Note that a method of forming the metal film is not particularly limited. For example, sputtering, plating, or the like may be used. As illustrated in FIG. 25, the shape of the diffraction grating 2111 is not particularly limited as long as plasmon resonance can be generated. For example, the diffraction grating 2111 may be a one-dimensional diffraction grating or a two-dimensional diffraction grating. For example, in the one-dimensional diffraction grating, a plurality of protrusions parallel to each other are formed at a predetermined interval on a surface of the metal film 2110. In the two-dimensional diffraction grating, protrusions having a predetermined shape are periodically arranged on the surface of the metal film 2110. Examples of the arrangement of the protrusions include a square lattice and a triangular (hexagonal) lattice. Examples of a cross-sectional shape of the diffraction grating 2111 include a rectangular waveform shape, a sinusoidal waveform shape, and a sawtooth shape. The surface of the metal film 2110 and the surface of the diffraction grating 2111 are surfaces treated with citric acid. That is, the surfaces of the plurality of capture regions 21b according to the present embodiment are configured by the surface of the metal film 2110 and the surface of the diffraction grating 2111. A method of forming the diffraction grating 2111 is not particularly limited. For example, after the metal film 2110 is formed on a flat substrate (not illustrated), an uneven shape may be imparted to the metal film 2110. In addition, the metal film 2110 may be formed on a substrate (not illustrated) to which an uneven shape is imparted in advance. In any method, the metal film 2110 including the diffraction grating 2111 can be formed.

In the present embodiment, the plurality of first isolators 22b and the plurality of second isolators 24a are formed of a light-responsive material. The light-responsive material forming the plurality of first isolators 22b and the plurality of second isolators 24a is a material that is hydrophilic when not irradiated with light of a predetermined wavelength by a stimulus applier 32a, and is a material that is hydrophobic when irradiated with light of a predetermined wavelength by the stimulus applier 32a. The predetermined wavelength is, for example, 360 nm in a case where the light-responsive material is an azobenzene-based compound. Since other configurations of the plurality of first isolators 22b and the plurality of second isolators 24a are equivalent to the configurations of the plurality of first isolators 22a and the plurality of second isolators 24 according to the second embodiment described above, the description thereof is omitted.

Since thin films of a light-responsive material are formed on the substrate body 200 as the plurality of first isolators 22b and the plurality of second isolators 24a, the stimulus applier 32a according to the present embodiment includes a light source or the like so that light can be applied to the light-responsive material. The stimulus applier 32a according to the present embodiment applies light to the first isolators 22b and the plurality of second isolators 24a of the analysis substrate 2b under control of a processing circuitry 37.

The light source 38 radiates excitation light to the analysis substrate 2b arranged in an analysis apparatus 3. The light source 38 emits light so that an incident angle with respect to the metal film 2110 becomes an angle that generates surface plasmon resonance. Here, the “excitation light” is light that directly or indirectly excites a fluorescent substance. For example, the excitation light is light that generates localized field light for exciting the fluorescent substance on the surface of the metal film 2110 when the metal film 2110 is irradiated with the excitation light through a prism 20 at an angle at which surface plasmon resonance occurs.

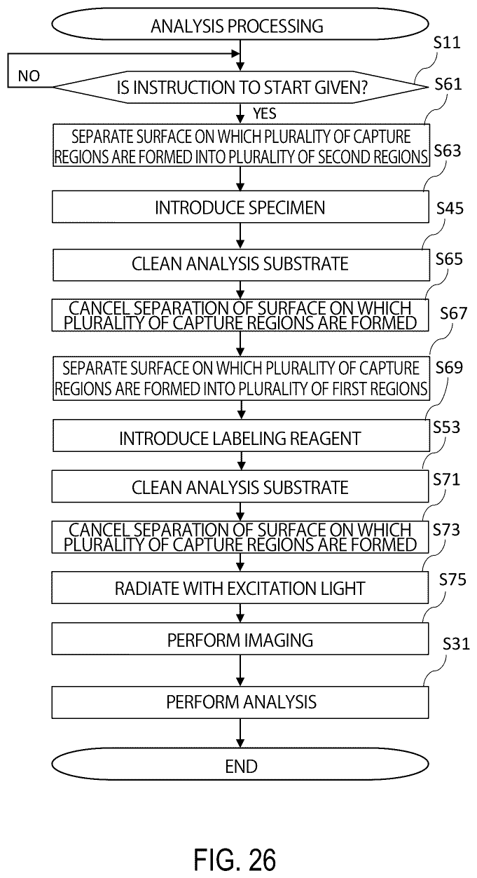

FIG. 26 is a flowchart for explaining contents of analysis processing performed by the analysis apparatus 3 according to the third embodiment, and is a view corresponding to FIG. 5 in the first embodiment described above. In this analysis processing, a stimulus is applied to the plurality of second isolators 24a of the analysis substrate 2b to introduce a specimen into the plurality of capture regions 21b, a stimulus is applied to the plurality of first isolators 22b to introduce a labeling reagent, a plurality of capture regions 21b of the analysis substrate 2b are imaged, or imaged images are analyzed. This analysis processing is processing executed in a case where an instruction to start analysis is given by a user. Note that the processing of Step S11 illustrated in FIG. 26 is equivalent to that in FIG. 5 in the first embodiment described above, and thus the description thereof is omitted.

On the other hand, in a case where an instruction to start analysis is given by the user (Step S11: Yes), the analysis apparatus 3 starts to apply a stimulus to the plurality of second isolators 24 (Step S61). The process of starting application of the stimulus to the plurality of second isolators 24 is realized by a control function 371 in the processing circuitry 37. Specifically, the analysis apparatus 3 separates a surface SU1 of the analysis substrate 2b on which the plurality of capture regions 21b are formed into the plurality of second regions AR21 to AR23 each including at least one capture region and provided along a second direction d2 intersecting with a first direction d1. More specifically, the analysis apparatus 3 according to the present embodiment controls a light source, which is a stimulus applier 32a, to apply light to the light-responsive material forming the plurality of second isolators 24a, such that the wettability of the plurality of second isolators 24a changes, and the surface SU1 of the analysis substrate 2b on which the plurality of capture regions 21b are formed is separated into the plurality of second regions AR21 to AR23.

Next, as illustrated in FIG. 26, the analysis apparatus 3 introduces a specimen SP (Step S63). The processing of introducing the specimen SP is realized by the control function 371 in the processing circuitry 37. Specifically, the analysis apparatus 3 introduces the specimen SP into the plurality of capture regions 21b by introducing the specimens SP different for each of the plurality of second regions AR21 to AR23 in a state where the wettability of the plurality of second isolators 24a is hydrophobic, that is, in a state where the isolators are separated into the plurality of second regions.

In the present embodiment, specimens SP1b to SP3b different for each of the plurality of second regions are specimens collected from different patients. For example, a specimen collected from a patient P1 is introduced into each of the capture regions 211b to 213b as the specimen SP1b. In addition, a specimen collected from a patient P2 is introduced into each of the capture regions 214b to 216b as the specimen SP2b. Furthermore, a specimen collected from a patient P3 is introduced into each of the capture regions 217b to 219b as the specimen SP3b. Note that, in Step S63, the specimen SP may be introduced onto the analysis substrate 2b automatically or manually. Since the description of Step S63 other than the above description is the same as the description of Step S43 in the second embodiment described above, the description thereof is omitted. In addition, since the processing of Step S45 after Step S63 is the same as that of Step S45 in the analysis processing of the second embodiment described above, the description thereof is omitted.

Next, as illustrated in FIG. 26, the analysis apparatus 3 cancels the separation of the surface SU1 on which the plurality of capture regions 21b are formed (Step S65). The process of canceling the separation of the surface SU1 on which the plurality of capture regions 21b are formed is realized by the control function 371 in the processing circuitry 37. Specifically, the analysis apparatus 3 controls the light source that is the stimulus applier 32a to stop the application of light to the light-responsive material forming the plurality of second isolators 24a, such that the wettability of the plurality of second isolators 24a returns to hydrophilicity. As a result, the separation of the surface SU1 of the analysis substrate 2b on which the plurality of capture regions 21b are formed is canceled.

Next, as illustrated in FIG. 26, the analysis apparatus 3 separates the surface SU1 on which the plurality of capture regions 21b are formed into a plurality of first regions (Step S67). The process of separating the surface SU1 on which the plurality of capture regions 21b are formed into the plurality of first regions is realized by the control function 371 in the processing circuitry 37. Specifically, the analysis apparatus 3 separates the surface SU1 of the analysis substrate 2b on which the plurality of capture regions 21b are formed into a plurality of first regions each including at least one capture region and provided along a first direction d1. More specifically, the analysis apparatus 3 according to the present embodiment controls a light source, which is a stimulus applier 32a, to apply light to the light-responsive material forming the plurality of first isolators 22b, such that the wettability of the plurality of first isolators 22b changes, and the surface SU1 of the analysis substrate 2b on which the plurality of capture regions 21b are formed is separated into the plurality of first regions. Since the description of Step S67 other than the above description is the same as the description of Step S49 in the second embodiment described above, the description thereof is omitted.

Next, as illustrated in FIG. 26, the analysis apparatus 3 introduces a labeling reagent (Step S69). The processing of introducing the labeling reagent is realized by the control function 371 in the processing circuitry 37. Specifically, the analysis apparatus 3 introduces labeling reagents different for each of the plurality of first regions AR11a to AR13a in a state where the wettability of the plurality of first isolators 22a is hydrophobic, that is, in a state where the first isolators 22a are separated into the plurality of first regions AR11a to AR13a. Note that, in Step S51, the labeling reagent may be introduced onto the analysis substrate 2b automatically or manually.

More specifically, in the present embodiment, the labeling reagents different for each of the plurality of first regions contain at least one common antibody. In addition, the labeling reagents different for each of the plurality of first regions labels EVs, which are fine particles, with fluorescent dyes different for each of the antibodies contained in the labeling reagents. For example, the labeling reagent introduced into each of the capture regions 211b, 214b, and 217b included in the first region contains an antibody that binds to BM1, an antibody that binds to BM2, an antibody that binds to BM3, and an antibody that binds to BM4 on the EVs captured in each of the capture regions 211b, 214b, and 217b, and biomarkers on the EVs are labeled with fluorescent dyes different for each of antibodies contained in the labeling reagent. The labeling reagent introduced into each of the capture regions 212b, 215b, and 218b included in the first region contains an antibody that binds to BM1, an antibody that binds to BM5, an antibody that binds to BM6, and an antibody that binds to BM7 on the EVs captured in each of the capture regions 212a, 215a, and 218a, and biomarkers on the EVs are labeled with fluorescent dyes different for each of the antibodies contained in the labeling reagent. Furthermore, the labeling reagent introduced into each of the capture regions 213a, 216a, and 219a included in the first region contains an antibody that binds to BM1, an antibody that binds to BM8, an antibody that binds to BM9, and an antibody that binds to BM10 on the EVs captured in each of the capture regions 213a, 216a, and 219a, and biomarkers on the EVs are labeled with fluorescent dyes different for each of the antibodies contained in the labeling reagent. As a result, it is possible to confirm a variation in population parameter for each of the plurality of first regions. Note that, in Step S69, the labeling reagent may be introduced onto the analysis substrate 2b automatically or manually. In addition, since the description of Step S53 after Step S63 is the same as that in the second embodiment described above, the description thereof is omitted.

Next, as illustrated in FIG. 26, the analysis apparatus 3 cancels the separation of the surface on which the plurality of capture regions 21b are formed (Step S71). The process of canceling the separation of the surface on which the plurality of capture regions 21b are formed is realized by the control function 371 in the processing circuitry 37. Specifically, the analysis apparatus 3 controls the light source that is the stimulus applier 32a to stop the application of light to the light-responsive material forming the plurality of first isolators 22b, such that the wettability of the plurality of first isolators 22b returns to hydrophilicity. As a result, the separation of the surface SU1 of the analysis substrate 2b on which the plurality of capture regions 21b are formed is canceled.

Next, as illustrated in FIG. 26, the analysis apparatus 3 radiates excitation light (Step S73). The processing of radiating the excitation light is realized by the control function 371 in the processing circuitry 37. Specifically, the analysis apparatus 3 controls the light source 38 to irradiate the plurality of capture regions 21b with excitation light.

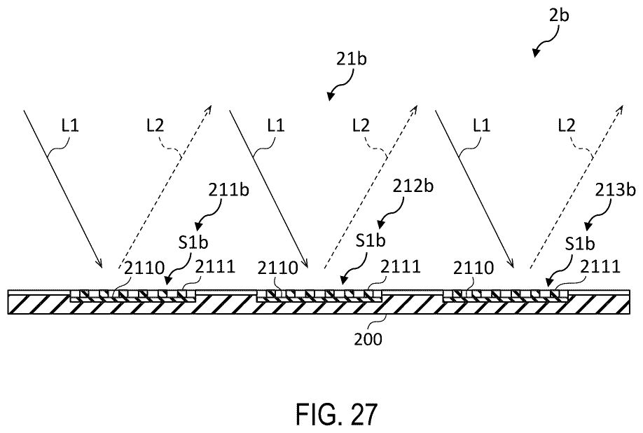

FIG. 27 is a view illustrating a state where the analysis apparatus 3 radiates excitation light to the analysis substrate 2b in the third embodiment. As illustrated in FIG. 27, in the present embodiment, when imaging is performed in Step S71, the analysis apparatus 3 controls the light source 38 to irradiate the metal film 2110 (diffraction grating 2111) with the excitation light L1 so that surface plasmon resonance occurs in the metal film 2110 (diffraction grating 2111). As a result, an electric field enhanced by surface plasmon resonance is generated in the vicinity of the metal film 2110 (diffraction grating 2111). Then, in a case where the biomarker labeled with the labeling reagent is captured on the metal films 2110 (diffraction gratings 2111) of the plurality of capture regions 21, the biomarker is excited by the enhanced electric field and emits fluorescence L2.

Next, as illustrated in FIG. 26, the analysis apparatus 3 performs imaging (Step S75). The processing of imaging is realized by the control function 371 in the processing circuitry 37. Specifically, the analysis apparatus 3 controls the imager 31 to image the plurality of capture regions 21 of the analysis substrate 2b. More specifically, an imager 31 images the plurality of capture regions 21 of the analysis substrate 2b to be irradiated with the excitation light L1, and surface plasmon resonance is generated in the plurality of capture regions 21b to detect an amplified fluorescence signal. In addition, since the description of Step S31 after Step S75 is the same as those in the first and second embodiments described above, the description thereof is omitted. Then, in Step S31, by analyzing the imaged image, the analysis processing according to the present embodiment is ended.

As described above, in the analysis substrate 2b according to the present embodiment, the metal films having a diffraction grating are provided on the plurality of capture regions 21b, and the metal film is irradiated with excitation light L1 by the light source 38 to generate the electric field enhanced by the surface plasmon resonance, such that EVs captured in the plurality of capture regions 21b and labeled with the labeling reagent emit fluorescence L2 due to the enhanced electric field. As a result, a fluorescence signal can be amplified, and the analysis efficiency can be improved. Therefore, a throughput of the fine particle analysis can be improved.

### Fourth Embodiment

In the analysis system 1 according to the first embodiment described above, the stimulus-responsive material is used as the flow path former 23 in the analysis substrate 2, but the flow path former 23 is not limited thereto. In a fourth embodiment, a flow path former 23 of an analysis substrate may be constituted by a wall. Hereinafter, parts different from the first to third embodiments described above will be described.

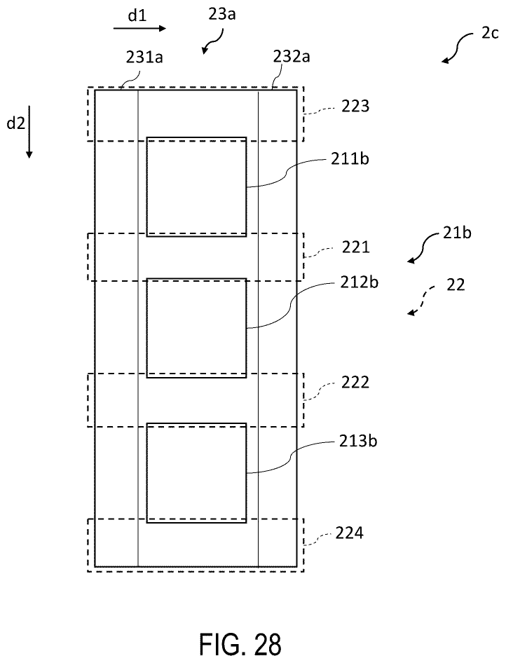

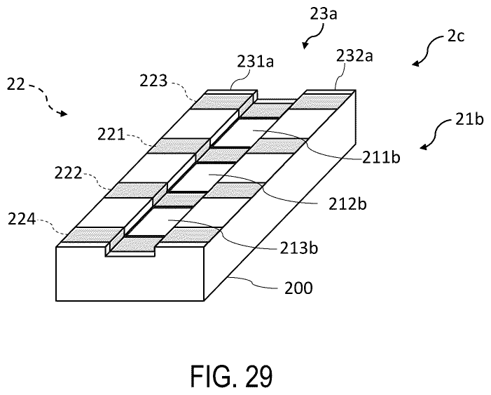

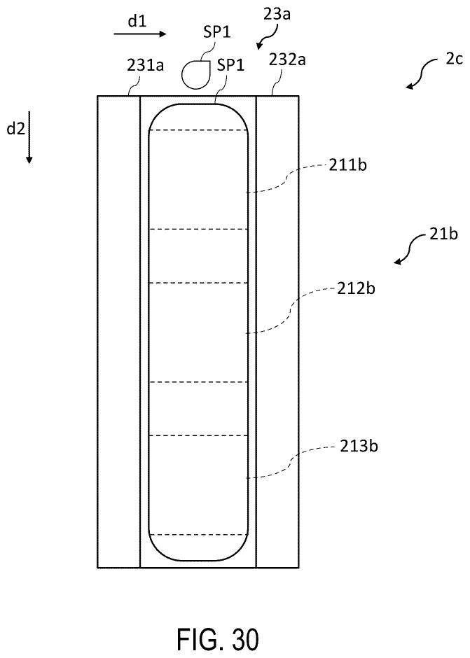

The analysis substrate according to the fourth embodiment will be described with reference to FIGS. 28 to 30. FIG. 28 is a top view illustrating an example of the analysis substrate according to the fourth embodiment, and is a view corresponding to FIG. 3 in the first embodiment described above. FIG. 29 is a perspective view illustrating an example of the analysis substrate in the fourth embodiment. FIG. 30 is a view illustrating an example of the analysis substrate onto which a specimen is introduced in the fourth embodiment. As illustrated in FIG. 28, the substrate body 200 according to the present embodiment includes a plurality of capture regions 21b, a plurality of first isolators 22, a flow path former 23a, and the substrate body 200. Note that, since a specific configuration of the plurality of capture regions 21b is the same as that in the third embodiment described above, the description thereof is omitted. In addition, since the configuration of the plurality of first isolators 22 and the substrate body 200 are the same as those in the first embodiment described above, the description thereof is omitted.

As illustrated in FIGS. 28 to 30, the flow path former 23a according to the present embodiment includes a first wall 231a and a second wall 232a. The first wall 231a is provided on the left side of each of a capture region 211b, a capture region 212b, and a capture region 213b. The second wall 232a is provided on the right side of each of the capture region 211b, the capture region 212b, and the capture region 213b. In addition, as illustrated in FIGS. 28 to 30, each of the first wall 231a and the second wall 232a is provided so as to transverse a surface of the substrate body 200 along a second direction d2. In addition, as illustrated in FIGS. 28 to 30, each of the first wall 231a and the second wall 232a is formed higher in a height direction than the surface of the analysis substrate 2c on which the plurality of capture regions 21b are formed. As a result, a flow path of a specimen is formed between the first wall 231a and the second wall 232a of the analysis substrate 2c. In a case where the analysis processing in the first embodiment described above is executed using the analysis substrate 2c including the flow path former 23a, an analysis apparatus 3 can introduce a specimen into the analysis substrate 2c in Step S15 without applying a stimulus to the flow path former 23a by the stimulus applier 32 in Step S13. Then, a specimen SP1 introduced into the analysis substrate 2c moves along the first wall 231a and the second wall 232a by the first wall 231a and the second wall 232a without falling from the left and right directions of the analysis substrate 2c.

As described above, in the analysis substrate 2c according to the present embodiment, the flow path former 23a includes the first wall 231a and the second wall 232a, and the flow path of the specimen SP1b is formed between the first wall 231a and the second wall 232a, such that the specimen SP1b can be introduced without applying a stimulus to the stimulus-responsive material, and a throughput of fine particle analysis can be improved.

### Fifth Embodiment

In the analysis systems 1 according to the second and third embodiments described above, in the analysis substrate 2, the stimulus-responsive material is used in the plurality of second isolators 24, but the plurality of second isolators 24 are not limited thereto. In a fifth embodiment, a plurality of second isolators of an analysis substrate may be constituted by walls. Hereinafter, parts different from the first to third embodiments described above will be described.

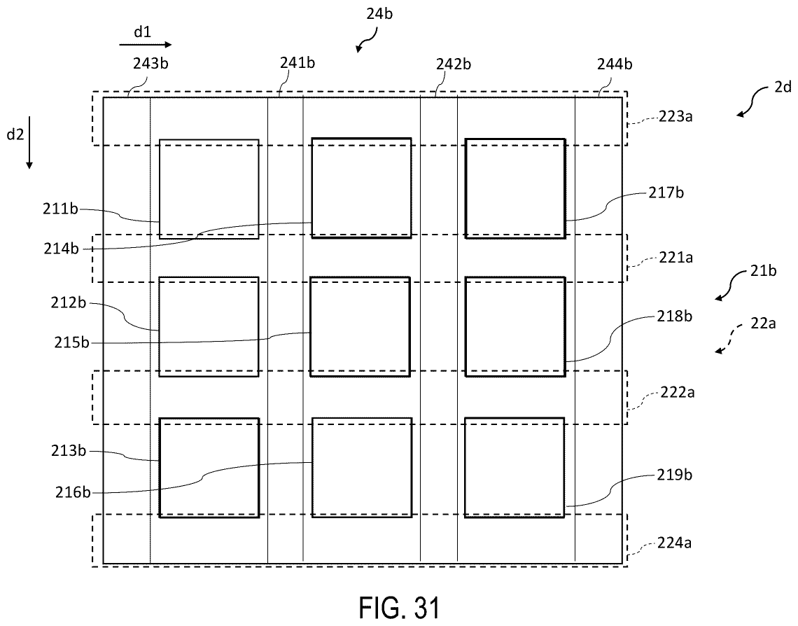

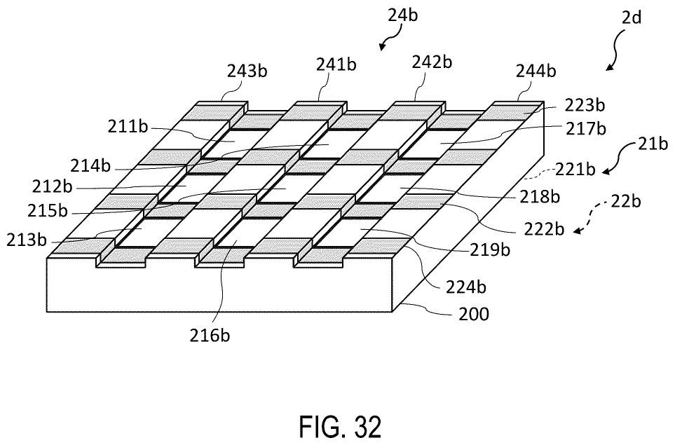

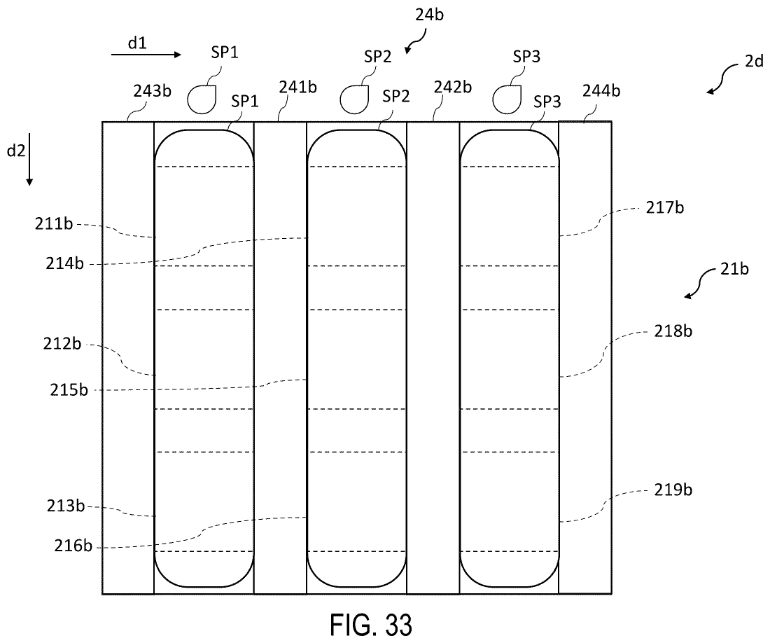

The analysis substrate according to the fifth embodiment will be described with reference to FIGS. 31 to 33. FIG. 31 is a top view illustrating an example of the analysis substrate according to the fifth embodiment, and is a view corresponding to FIG. 14 in the second embodiment described above. FIG. 32 is a perspective view illustrating an example of the analysis substrate according to the fifth embodiment. FIG. 33 is a view illustrating an example of the analysis substrate onto which a specimen is introduced in the fifth embodiment, and is a view corresponding to FIG. 18 in the second embodiment described above. As illustrated in FIG. 31, the analysis substrate 2d according to the present embodiment includes a plurality of capture regions 21b, a plurality of first isolators 22a, a plurality of second isolators 24b, and the substrate body 200. Note that, since a specific configuration of the plurality of capture regions 21b is the same as that in the third embodiment described above, the description thereof is omitted. In addition, since the configuration of the plurality of first isolators 22a is the same as that in the second embodiment described above, the description thereof is omitted. In addition, since the configuration of the substrate body 200 is the same as that in the first embodiment described above, the description thereof is omitted.

The plurality of second isolators 24b according to the present embodiment are partition walls that separate the plurality of capture regions 21b. Specifically, as illustrated in FIGS. 31 and 32, the plurality of second isolators 24b according to the present embodiment include a first partition wall 241b, a second partition wall 242b, a third partition wall 243b, and a fourth partition wall 244b. The first partition wall 241b is a wall provided along a first direction d1 between capture regions 211b to 213b and capture regions 214b to 216b. That is, the first partition wall 241b is a plurality of capture regions. The first partition wall 241b is a partition wall that separates the capture regions 211b to 213b and the capture regions 214b to 216b. The second partition wall 242b is a wall provided along the first direction d1 between the capture regions 214b to 216b and capture regions 216b to 219b. That is, the second partition wall 242b is a partition wall that separates the capture regions 214b to 216b and the capture regions 216b to 219b. The third partition wall 243b is a wall provided along the first direction d1 on a side opposite to the first partition wall 241b with respect to the capture regions 211b to 213b. The fourth partition wall 244b is a wall provided along the first direction d1 on a side opposite to the second partition wall 242b with respect to the capture regions 216b to 219b.

As illustrated in FIG. 33, the plurality of second isolators 24b of the analysis substrate 2d includes the first partition wall 241b and the third partition wall 243b, such that a flow path for introducing a specimen SP1 into the capture regions 211b to 213b is formed between the first partition wall 241b and the third partition wall 243b. In addition, the plurality of second isolators 24b of the analysis substrate 2d includes the first partition wall 241b and the third partition wall 243b, such that a flow path for introducing a specimen SP2 into the capture regions 214b to 216b is formed between the first partition wall 241b and the second partition wall 242b. Furthermore, the plurality of second isolators 24b of the analysis substrate 2d includes the second partition wall 242b and the fourth partition wall 244b, such that a flow path for introducing a specimen SP3 into the capture regions 216b to 219b is formed between the second partition wall 242b and the fourth partition wall 244b. Then, in a case where the analysis processing in the second embodiment described above is executed using the analysis substrate 2d including the plurality of second isolators 24b, in Step S41, application of a stimulus by a stimulus applier 32 does not start, and as illustrated in FIG. 33, the analysis apparatus 3 can introduce a specimen onto the analysis substrate 2d in Step S43. That is, the first to fourth partition walls 231b to 234b are provided, such that specimens SP1 to SP3 introduced into a second region AR21, a second region AR22, and a second regions AR23, respectively, can be introduced without being mixed.

As described above, in the analysis substrate 2d according to the present embodiment, the plurality of second isolators 24b include the first partition wall 241b, the second partition wall 242b, the third partition wall 243b, and the fourth partition wall 244b, and flow paths are formed between the first partition wall 241b and the third partition wall 243b, between the first partition wall 241b and the second partition wall 242b, and between the second partition wall 242b and the fourth partition wall 244b, respectively. Therefore, the specimens SP1 to SP3 introduced into the second region AR21, the second region AR22, and the second region AR23 can be introduced without mixing. Therefore, a throughput of the fine particle analysis can be improved.

### Sixth Embodiment

In the second, third, and fifth embodiments described above, the analysis substrate includes the plurality of first isolators and the plurality of second isolators, such that the plurality of labeling reagents or plurality of specimens are introduced into the analysis substrate without being mixed. However, the method of introducing the plurality of labeling reagents or plurality of specimens into the analysis substrate without mixing them is not limited to the case where the analysis substrate includes a plurality of first isolators or a plurality of second isolators. In an analysis system according to a sixth embodiment, when a plurality of specimens or a plurality of labeling reagents are introduced into an analysis substrate, a separator for separating each of a plurality of capture regions may be arranged on the analysis substrate. Hereinafter, parts different from the first to fifth embodiments described above will be described.

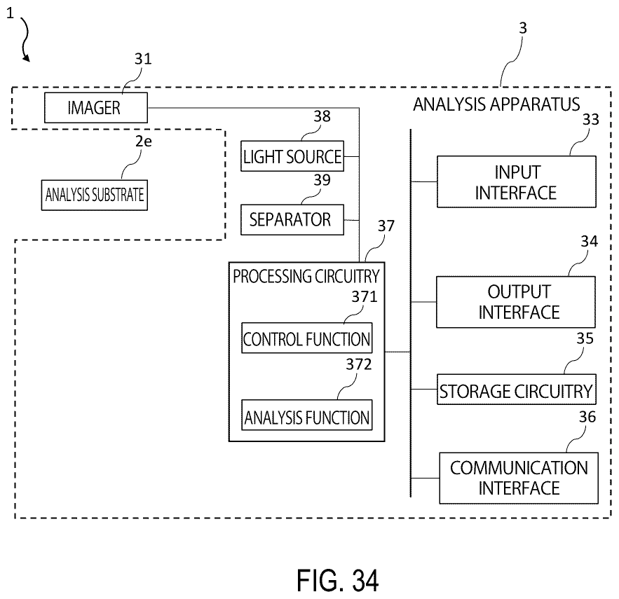

FIG. 34 is a block diagram illustrating an example of a configuration of an analysis system 1 according to the sixth embodiment, and is a view corresponding to FIG. 23 in the third embodiment described above. As illustrated in FIG. 34, since a configuration of an analysis substrate 2e is different from that of the third embodiment, in the present embodiment, it is referred to as a analysis substrate 2e. In addition, an analysis system 1 according to the present embodiment is configured by adding a separator 39 to the analysis system 1 according to the third embodiment.

Furthermore, the analysis system 1 according to the present embodiment does not include a stimulus applier 32 unlike the analysis system 1 according to the third embodiment. Since configurations and functions other than the analysis substrate 2e, the stimulus applier 32, and the separator 39 are the same as those in FIG. 23 in the third embodiment described above, the description thereof is omitted.

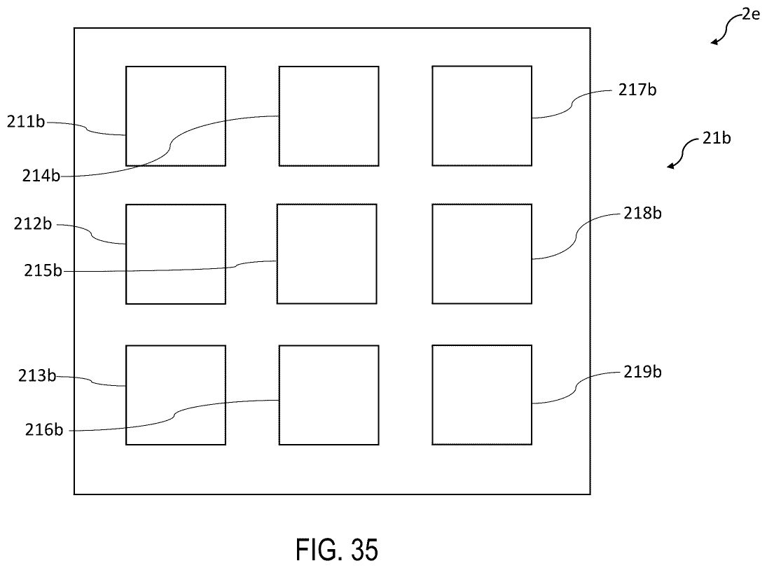

The analysis substrate 2e according to the present embodiment will be described with reference to FIG. 35. FIG. 35 is a top view illustrating an example of the analysis substrate 2e according to the present embodiment, and is a view corresponding to FIG. 23 in the third embodiment described above. As illustrated in FIG. 35, the analysis substrate 2e according to the present embodiment includes a plurality of capture regions 21b and the substrate body 200. That is, the analysis substrate 2e according to the present embodiment is different from the analysis substrate 2b according to the third embodiment in that a plurality of first isolators 22b and a plurality of second isolators 24a are not provided. Note that, since a specific configuration of the plurality of capture regions 21b is the same as that in the third embodiment described above, the description thereof is omitted. In addition, since the configuration of the substrate body 200 are the same as that in the first embodiment described above, the description thereof is omitted.

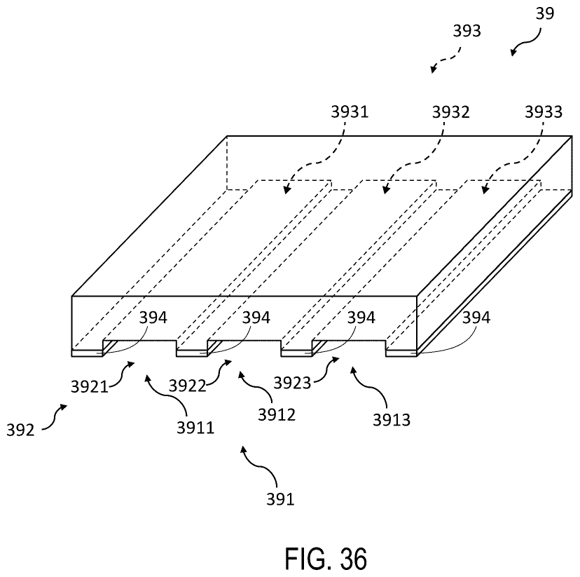

The separator 39 is a member for separating surfaces of the plurality of capture regions 21b of the analysis substrate 2e into a plurality of regions. The separator 39 is arranged on the surface of the analysis substrate 2e including the plurality of capture regions 21b, such that a flow path for introducing a specimen or a labeling reagent into each of the plurality regions of the analysis substrate 2e is defined. The separator 39 is formed of, for example, glass or a transparent resin. For example, the separator 39 is arranged on the analysis substrate 2e or removed from the analysis substrate 2e under control of a processing circuitry 37. FIG. 36 is a perspective view illustrating an example of the separator 39 according to the present embodiment. As illustrated in FIG. 36, three grooves 3911 to 3913 are formed in the bottom of the separator 39. In a case where the separator 39 is arranged on the analysis substrate 2e, the three grooves 3911 to 3913 serve as flow paths through which a specimen or a labeling reagent flows. In the example illustrated in FIG. 36, the separator 39 is provided with inflow ports 3921 to 3923 and outflow ports 3931 to 3933 for introducing a specimen and a labeling reagent that communicate with the three grooves 3911 to 3913, respectively. Furthermore, as illustrated in FIG. 36, an elastic member 394 is provided at the bottom of the separator 39. In a case where the separator 39 is arranged on the analysis substrate 2e, the elastic member 394 is in close contact with a portion of an upper surface of the substrate body 200 other than the plurality of capture regions 21b. As a result, in a case where a specimen or a labeling reagent is introduced into the analysis substrate 2e, the specimen or the labeling reagent is prevented from flowing into another flow path into the analysis substrate 2e.

Note that, although the three grooves 3911 to 3913 are formed at the bottom of the separator 39 according to the present embodiment, the number of grooves formed at the bottom of the separator 39 is not limited to three. That is, the number of grooves formed at the bottom of the separator 39 is arbitrary, and two grooves may be formed, or four or more grooves may be formed. The number of grooves may be determined according to the mode of the plurality of capture regions 21b of the analysis substrate 2e.

In addition, the separator 39 according to the present embodiment is formed of glass, a transparent resin, or the like, but the material of the separator 39 is not limited thereto. That is, the material of the separator 39 is arbitrary, and may be formed of, for example, an opaque resin or a metal. Note that, in a case where the separator 39 is formed of an elastic member, the elastic member 394 may not be provided at the bottom of the separator 39.

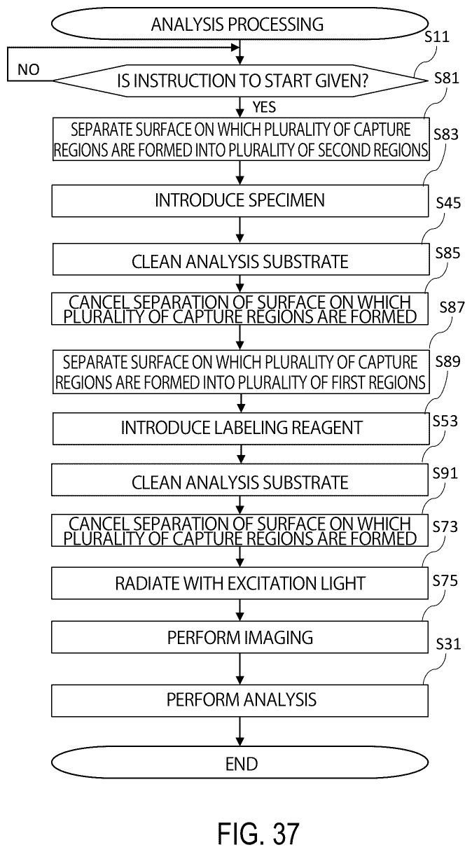

FIG. 37 is a flowchart for explaining contents of analysis processing performed by an analysis apparatus 3 according to the sixth embodiment, and is a view corresponding to FIG. 26 in the third embodiment described above. In this analysis processing, the separator 39 is arranged along a first direction and a specimen is introduced into the plurality of capture regions 21b, the separator 39 is arranged in a second direction and a labeling reagent is introduced into the plurality of capture regions 21b, the plurality of capture regions 21 of the analysis substrate 2e are imaged, or the imaged images are analyzed. This analysis processing is processing executed in a case where an instruction to start analysis is given by a user. Note that the processing of Step S11 illustrated in FIG. 26 is equivalent to that in FIG. 5 in the first embodiment described above, and thus the description thereof is omitted.

Next, as illustrated in FIG. 37, the analysis apparatus 3 separates a surface SU1 on which the plurality of capture regions 21b are formed into a plurality of second regions (Step S81). The process of separating the surface SU1 on which the plurality of capture regions 21b are formed into the plurality of second regions is realized by the control function 371 in the processing circuitry 37. Specifically, the separator 39 is arranged on the analysis substrate 2e, such that the surface SU1 on which the plurality of capture regions 21b are formed is separated into the plurality of second regions.

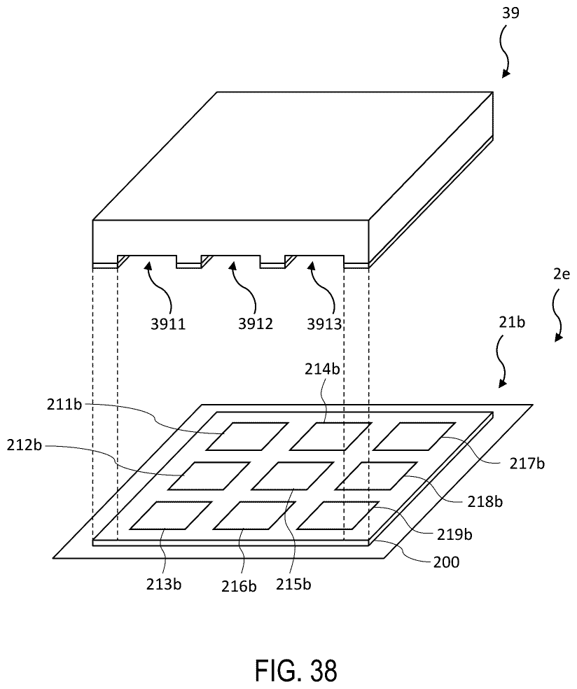

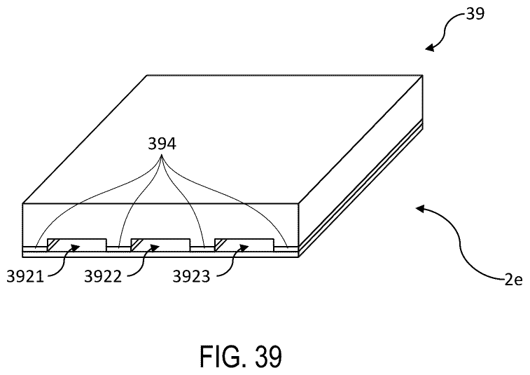

The arrangement of the separator 39 in Step S81 will be described in detail with reference to FIGS. 38 and 39. FIG. 38 is a view for explaining a positional relationship between the separator 39 and the analysis substrate 2e at the time of introducing the specimen in the present embodiment. FIG. 39 is a view illustrating a state where the separator 39 is arranged on the analysis substrate 2e at the time of introducing the specimen in the present embodiment. As illustrated in FIG. 38, the groove 3911 of the separator 39 according to the present embodiment corresponds to capture regions 211b to 213b and serves as a flow path for introducing a specimen into the capture regions 211b to 213b. In addition, the groove 3912 of the separator 39 according to the present embodiment corresponds to capture regions 214b to 216b and serves as a flow path for introducing a specimen into the capture regions 214b to 216b. The groove 3913 of the separator 39 according to the embodiment corresponds to capture regions 217b to 219b and serves as a flow path for introducing a specimen into the capture regions 217b to 219b. As illustrated in FIG. 39, the separator 39 is arranged on the analysis substrate 2e so that a longitudinal direction of each of the three grooves 3911 to 3911 of the separator 39 is along the first direction d1. At this time, the bottom of the separator 39 is arranged in contact with the analysis substrate 2e so as not to overlap the plurality of capture regions 21b of the analysis substrate 2e.

Next, as illustrated in FIG. 37, the analysis apparatus 3 introduces a specimen (Step S83). The processing of introducing the specimen is realized by the control function 371 in the processing circuitry 37. Specifically, the analysis apparatus 3 introduces a specimen into the plurality of capture regions 21b from inflow ports 3921 to 3923 of the separator 39, respectively. In the present embodiment, a specimen SP1b is introduced into the capture regions 211b to 213b, a specimen SP2b is introduced into the capture regions 214b to 216b, and a specimen SP3b is introduced into the capture regions 217b to 219b. When these specimens SP1b to SP3b are introduced, the specimen SP1b is introduced into the capture regions 211b to 213b from the inflow port 3921, the specimen SP2b is introduced into the capture regions 214b to 216b from the inflow port 3922, and the specimen SP3b is introduced into the capture regions 217b to 219b from the inflow port 3923. Note that, in Step S83, the specimen may be introduced onto the analysis substrate 2e automatically or manually. In addition, since the description of Step S45 after Step S83 is the same as that in the second embodiment described above, the description thereof is omitted.

Next, as illustrated in FIG. 37, the analysis apparatus 3 cancels the separation of the surface SU1 on which the plurality of capture regions 21b are formed (Step S87). The process of canceling the separation of the surface on which the plurality of capture regions 21b are formed is realized by the control function 371 in the processing circuitry 37. Specifically, the analysis apparatus 3 moves the separator 39 upward and removes the separator 39 from the analysis substrate 2e, such that the separation of the surface SU1 on which the plurality of capture regions 21b are formed is canceled.

Next, as illustrated in FIG. 37, the analysis apparatus 3 separates a surface SU1 on which the plurality of capture regions 21b are formed into a plurality of first regions (Step S81). The process of separating the surface SU1 on which the plurality of capture regions 21b are formed into the plurality of second regions is realized by the control function 371 in the processing circuitry 37. Specifically, the analysis apparatus 3 separates the surface SU1 on which the plurality of capture regions 21b are formed into the plurality of first regions by arranging the separator 39 on the analysis substrate 2e.

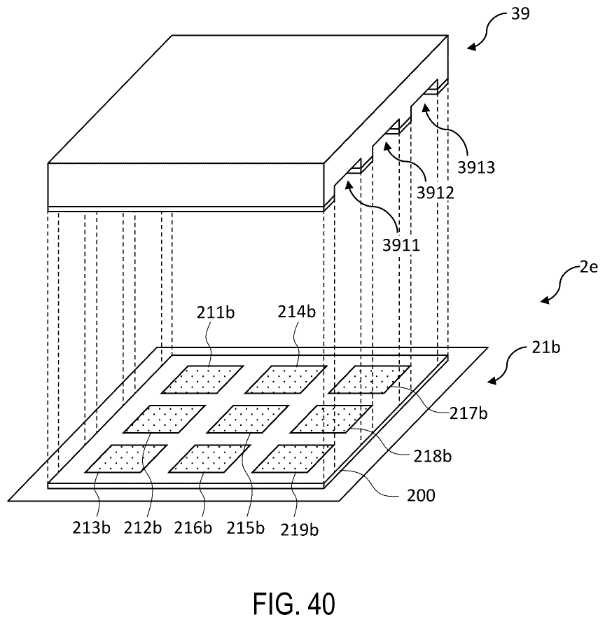

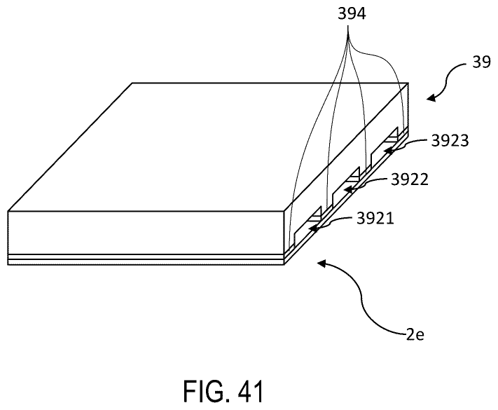

The arrangement of the separator 39 in Step S87 will be described in detail with reference to FIGS. 40 and 41. FIG. 40 is a view for explaining a positional relationship between the separator 39 and the analysis substrate 2e at the time of introducing the labeling reagent in the present embodiment. FIG. 41 is a view illustrating a state where the separator 39 is arranged on the analysis substrate 2e at the time of introducing the labeling reagent in the present embodiment. As illustrated in FIG. 40, the separator 39 according to the present embodiment is arranged above the analysis substrate 2e by rotating the separator 39 by 90 degrees about a line perpendicular to the upper surface of the separator 39 with respect to the position of the separator 39 when the specimen is introduced. As a result, the groove 3911 of the separator 39 corresponds to the capture regions 213b, 216b, and 219b and serves as a flow path for introducing a labeling reagent into the capture regions 213b, 216b, and 219b. In addition, the groove 3912 of the separator 39 according to the present embodiment corresponds to the capture regions 212b, 215b, and 218b and serves as a flow path for introducing a labeling reagent into the capture regions 212b, 215b, and 218b. Furthermore, the groove 3913 of the separator 39 according to the present embodiment corresponds to the capture regions 211b, 214b, and 217b and serves as a flow path for introducing a labeling reagent into the capture regions 211b, 214b, and 217b. As illustrated in FIG. 41, the separator 39 is arranged on the analysis substrate 2e so that a longitudinal direction of each of the three grooves 3911 to 3911 of the separator 39 is along the second direction d2. At this time, the bottom of the separator 39 is arranged in contact with the analysis substrate 2e so as not to overlap the plurality of capture regions 21b of the analysis substrate 2e.

Next, as illustrated in FIG. 37, the analysis apparatus 3 introduces a labeling reagent (Step S89). The processing of introducing the labeling reagent is realized by the control function 371 in the processing circuitry 37. Specifically, the analysis apparatus 3 introduces labeling reagents LR1 to LR3 into the plurality of capture regions 21b from the inflow ports 3921 to 3923 of the separator 39, respectively. In the present embodiment, the labeling reagent LR1 is introduced into the capture regions 211b, 214b, and 217b, the labeling reagent LR2 is introduced into the capture regions 212b, 215b, and 218b, and the labeling reagent LR3 is introduced into the capture regions 213b, 216b, and 219b. In a case where these labeling reagents LR1 to LR3 are introduced, the labeling reagent LR1 is introduced into the capture regions 211b, 214b, and 217b from the inflow port 3923, the labeling reagent LR2 is introduced into the capture regions 212b, 215b, and 218b from the inflow port 3922, and the labeling reagent LR3 is introduced into the capture regions 213b, 216b, and 219b from the inflow port 3923. Note that, in Step S89, the labeling reagent may be introduced onto the analysis substrate 2e automatically or manually. In addition, since the description of Step S53 after Step S89 is the same as that in the second embodiment described above, the description thereof is omitted.

Next, as illustrated in FIG. 37, the analysis apparatus 3 cancels the separation of the surface SU1 on which the plurality of capture regions 21b are formed (Step S91). The process of canceling the separation of the surface SU1 on which the plurality of capture regions 21b are formed is realized by the control function 371 in the processing circuitry 37. Specifically, the analysis apparatus 3 moves the separator 39 upward and removes the separator 39 from the analysis substrate 2e, such that the separation of the surface SU1 on which the plurality of capture regions 21b are formed is canceled. In addition, since the description of Step S73 and Step S31 after Step S91 is the same as those in the first to third embodiments described above, the description thereof is omitted. Then, in Step S31, by analyzing the imaged image, the analysis processing according to the present embodiment is ended.

As described above, in the analysis system 1 according to the present embodiment, the separator 39 isolates the plurality of capture regions 21b into a plurality of capture region groups, and the separator 39 is arranged on the analysis substrate 2e, such that a flow path for introducing a specimen or a labeling reagent into each of the plurality of first regions and second regions of the analysis substrate 2e is defined. Therefore, when the specimen or the labeling reagent is introduced into the analysis substrate 2e without forming the plurality of first isolators and the plurality of second isolators for isolating the plurality of capture regions on the analysis substrate 2e, each of the specimens or each of the labeling reagents can be prevented from being mixed. That is, on one analysis substrate 2e, a plurality of biomarkers in a plurality of specimens can be simultaneously analyzed, such that the analysis efficiency can be improved, and a throughput of the fine particle analysis can be improved.

Note that, in the sixth embodiment described above, the analysis apparatus 3 rotates the separator 39 to introduce a specimen and a labeling reagent, but the analysis substrate 2e may be rotated without rotating the separator 39 to introduce the specimen and the labeling reagent. In addition, in the sixth embodiment described above, the analysis apparatus 3 includes one separator and introduces a specimen and a labeling reagent by rotating the separator 39, but the present disclosure is not limited thereto. The analysis apparatus 3 includes two separators 39, and the two separators may be selectively used for introduction of a specimen and introduction of a labeling reagent.

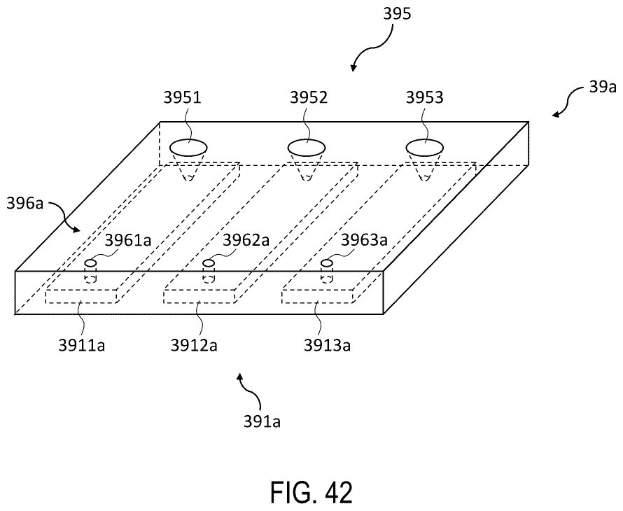

In addition, in the sixth embodiment described above, the separator 39 is provided with inflow ports 3921 to 3923 and outflow ports 3931 to 3933 for introducing a specimen and a labeling reagent that communicate with the three grooves 3911 to 3913, respectively, and the configuration of the separator 39 is not limited thereto. FIG. 42 is a view illustrating another example of the separator 39 according to the sixth embodiment. As illustrated in FIG. 42, a separator 39a may be provided with three grooves 3911a to 3913a formed at the bottom of the separator 39a, dropping ports 3951 to 3953 that communicate with the grooves 3911a to 3913a, respectively, from an upper portion of the separator 39, and introduce a specimen into each of the grooves 3911a to 3913a, and air holes 3961 to 3963 that are provided at positions different from those of the dropping ports 3951 to 3953 and communicate with the grooves 3911a to 3913a from the upper portion of the separator 39. In addition, the dropping ports 3951 to 3953 may be inclined so that a diameter decreases from the upper portion of the separator 39 toward each of the grooves 3911a to 3913a. Even when such a separator 39a is used, the same effects as those of the sixth embodiment can be expected.

### Modifications of First to Sixth Embodiments

In the first to sixth embodiments described above, in a case where a specimen is introduced into the plurality of capture regions 21, 21a, and 21b, a spotter or a dispenser can also be used. Hereinafter, parts different from the first to sixth embodiments described above will be described.

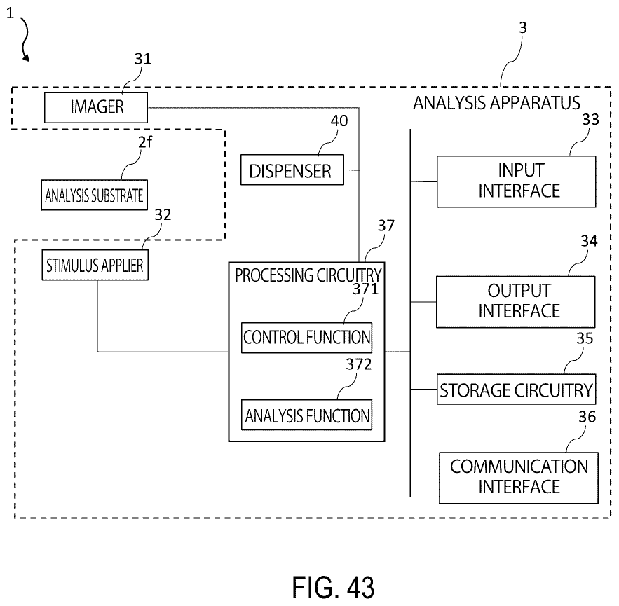

FIG. 43 is a view illustrating an example of a configuration of an analysis system 1 according to a first modification, and is a view corresponding to FIG. 2 in the first embodiment described above. As illustrated in FIG. 43, since a configuration of an analysis substrate is different from that of the first embodiment, in the present embodiment, it is referred to as a analysis substrate 2f. As illustrated in FIG. 43, an analysis apparatus 3 in the analysis system 1 according to the present modification is configured to add a dispenser 40 to the analysis apparatus 3 according to the second embodiment described above. Since configurations and functions other than analysis the substrate 2f and the dispenser 40 are the same as those in FIG. 2 in the first embodiment described above, the description thereof is omitted.

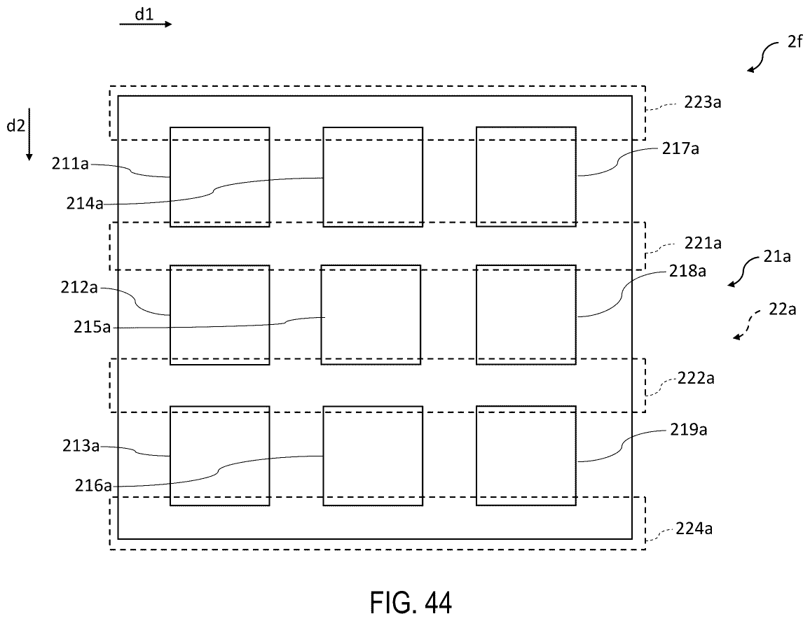

FIG. 44 is a top view illustrating an example of a configuration of the analysis substrate 2f according to the first modification, and is a view corresponding to FIG. 13 in the second embodiment described above. As illustrated in FIG. 13, the analysis substrate 2f according to the present embodiment differs in that it does not include the plurality of second isolators 24. Since the other configurations of the analysis substrate 2f are the same as those of the analysis substrate 2a according to the second embodiment described above, the description thereof is omitted.

The dispenser 40 dispenses a specimen to each of a plurality of capture regions 21a under control of a processing circuitry 37. In the present embodiment, the dispenser 40 uses different nozzles for each specimen to be dispensed into the plurality of capture regions 21a.

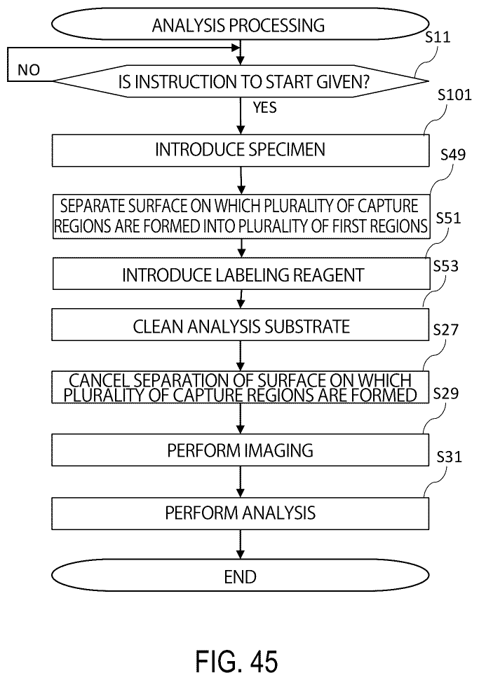

FIG. 45 is a flowchart for explaining contents of analysis processing performed by the analysis apparatus 3 according to the first modification. In this analysis processing, a specimen is introduced into the plurality of capture regions 21a, a stimulus is applied to a plurality of first isolators 22 to introduce a labeling reagent, the plurality of capture regions 21a of the analysis substrate 2f are imaged, or imaged images are analyzed. This analysis processing is processing executed in a case where an instruction to start analysis is given by a user. Note that the processing of Step S11 illustrated in FIG. 45 is equivalent to that in FIG. 15 in the second embodiment described above, and thus the description thereof is omitted.

Next, as illustrated in FIG. 45, the analysis apparatus 3 introduces a specimen (Step S101). The processing of introducing the specimen is realized by the control function 371 in the processing circuitry 37. Specifically, the analysis apparatus 3 controls the dispenser 40 to introduce a specimen into each of the plurality of capture regions 21a.

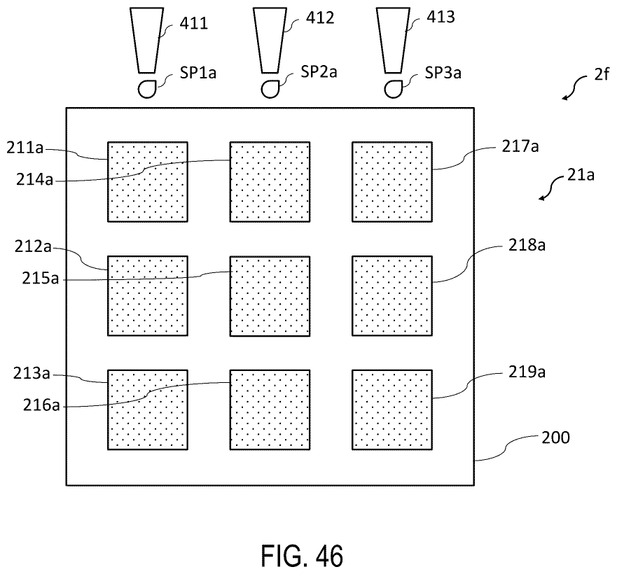

FIG. 46 is a view illustrating an example of an analysis substrate 2f onto which a specimen is introduced in an analysis system 1 according to the present modification. As illustrated in FIG. 46, in the present modification, similarly to the second embodiment, a specimen diluted to have a concentration of X % is introduced as a specimen SP1a into capture regions 211a to 213a included in a second region AR21. In addition, a specimen diluted to have a concentration of Y % is introduced as the specimen SP2a into the capture regions 214a to 216a included in the second region AR22. Furthermore, a specimen diluted to have a concentration of Z % is introduced as the specimen SP3a into the capture regions 217a to 219a included in the second region AR23. In addition, in the present modification, the dispenser 40 uses different nozzles for each specimen. Therefore, as illustrated in FIG. 46, the analysis apparatus 3 controls a nozzle 411 of the dispenser 40 to introduce the specimen SP1a into the capture regions 211a to 213a, controls a nozzle 412 of the dispenser 40 to introduce the specimen SP2a into the capture regions 214a to 216a, and controls a nozzle 413 of the dispenser 40 to introduce the specimen SP3a into the capture regions 217a to 219a. Therefore, the specimens SP1a to SP3a can be introduced into the plurality of capture regions 21a, respectively, while reducing a risk of mixing the specimen SP1a to SP3a.

Note the, since the processing from Step S49 after Step S101 is the same as the processing in FIG. 15 in the second embodiment described above, the description thereof is omitted. Then, in Step S31, by analyzing the imaged image, the analysis processing according to the present embodiment is ended.

As described above, in the analysis system 1 according to the present modification, the analysis apparatus 3 includes the dispenser 40 and a specimen is introduced onto the analysis substrate 2f using the dispenser 40, it is possible to introduce the specimen onto the analysis substrate 2f while preventing the specimen from being mixed without changing wettability of the plurality of second isolators 24.

In addition, in the analysis system 1 according to the present modification, since the analysis apparatus 3 includes the dispenser 40 and introduces a specimen onto the analysis substrate 2f using the dispenser 40, it is not necessary to clean the analysis substrate 2f by introduction of the specimen. Therefore, a throughput of the fine particle analysis can be further improved.

Note that, in the first modification described above, the analysis apparatus 3 includes the dispenser 40, but may include a spotter instead of the dispenser 40. Even in a case where a spotter is provided, the same effect as that of the first modification can be expected. Furthermore, in the first modification, the dispenser 40 uses different nozzles for each specimen to be dispensed into the plurality of capture regions 21a, but the same nozzle may be used for each specimen to be dispensed into the plurality of capture regions 21a. As described above, in a case where the same nozzle is used, the nozzle may be cleaned before dispensing different specimens.

### Second Modification of First to Sixth Embodiments

In the first to sixth embodiments described above, polyethylene glycol is bonded to surfaces of the plurality of capture regions 21, 21a, and 21b via N-hydroxysuccinimide, the surfaces of the plurality of capture regions 21 are treated with citric acid, or fine particles are captured non-specifically by covalent bonding, physical adsorption, chemical adsorption, or the like. However, the fine particles may be captured by immobilizing antibodies for capturing the fine particles on the surfaces of the plurality of capture regions 21, 21a, and 21b.

In addition, in a case where the fine particles are captured with the antibodies, different types of antibodies may be supported for each of the plurality of capture regions 21, 21a, and 21b. For example, different types of antibodies may be carried (immobilized) for each row of the plurality of capture regions 21, 21a, and 21b. Also, a mixture of antibodies may be immobilized in each capture region.

### Other Modifications of First to Sixth Embodiments

In the first to sixth embodiments and the first and second modifications described above, the first isolator and/or second isolator included in the analysis substrates 2 and 2a to 2f is formed using a stimulus-responsive material whose wettability changes from hydrophilic to hydrophobic in response to a stimulus. However, the first isolator and/or the second isolator may be formed using a stimulus-responsive material whose wettability changes from hydrophobic to hydrophilic in response to a stimulus.

In addition, in the second, third, and sixth embodiments described above, the analysis substrates 2a, 2b, and 2d provided between the plurality of second isolators 24, 24a, and 24b provided between the capture regions along the second direction d2 intersecting with the first direction d1. However, the number of second isolators provided between the capture regions along the second direction d2 intersecting with the first direction d1 may be one. In a case where the analysis substrate includes one second isolator, for example, the analysis substrate may include six capture regions, and may be arranged in three rows and two columns on the substrate body 200.

In addition, in the second, third, and sixth embodiments described above, the analysis substrates 2a, 2b, and 2d are used to analyze a plurality of specimens, but the analysis substrates 2a, 2b, and 2d may be used to analyze one specimen.

In addition, in the first to sixth embodiments and the first and second modifications described above, the shape of each of the plurality of capture regions 21, 21a, and 21b is rectangular, but the shape of each of the plurality of capture regions 21, 21a, and 21b is not limited thereto. That is, the shape of each of the plurality of capture regions 21, 21a, and 21b is arbitrary, and may be, for example, a circular shape, an elliptical shape, or the like. In addition, in the example illustrated in FIG. 4, the plurality of capture regions 21, 21a, and 21b are formed so as to be recessed from the surfaces of the substrate body 200, but the plurality of capture regions 21, 21a, and 21b may be formed on the surface of the substrate body 200.

In addition, in the first to sixth embodiments and the first and second modifications described above, imaging is performed after the separation of the surface on which the plurality of capture regions are formed is canceled, but imaging may be performed without canceling the separation of the surface on which the plurality of capture regions are formed.

Note that the term “processor” used in the above description means, for example, a central processing unit (CPU), a graphics processing unit (GPU), or a circuit such as an application specific integrated circuit (ASIC) or a programmable logic device (for example, a simple programmable logic device (SPLD), a complex programmable logic device (CPLD), or a field programmable gate array (FPGA)). The processor realizes the function by reading and executing the program stored in the memory 41. Note that, instead of storing the program in the storage circuitry 35, the program may be directly incorporated in the circuit of the processor. In this case, the processor realizes the function by reading and executing the program incorporated into the circuit. Note that the processor is not limited to a case of being configured as a single processor circuit, and a plurality of independent circuits may be combined to be configured as one processor to realize the function. Furthermore, a plurality of components in FIGS. 2, 23, 34, and 43 may be integrated into one processor to realize the function.

Although several embodiments have been described above, these embodiments have been presented only as examples, and are not intended to limit the scope of the invention. The novel apparatuses and methods described in the present specification can be implemented in various other forms. In addition, various omissions, substitutions, and changes can be made to the forms of the apparatus and the method described in the present specification without departing from the gist of the invention. The appended claims and their equivalents are intended to include such forms and modifications as fall within the scope and spirit of the invention.

1. An analysis substrate for performing analysis on fine particles, the analysis substrate comprising:
a plurality of capture regions configured to capture the fine particles; and
a plurality of first isolators whose wettability changes in response to a stimulus, the plurality of first isolators being provided along a first direction between the plurality of capture regions.

2. The analysis substrate according to claim 1, further comprising a second isolator provided between the plurality of capture regions along a second direction intersecting with the first direction.

3. The analysis substrate according to claim 2, wherein the second isolator is provided between the plurality of capture regions along the second direction orthogonal to the first direction.

4. The analysis substrate according to claim 2, wherein wettability of the second isolator changes in response to a stimulus.

5. The analysis substrate according to claim 1, wherein the plurality of first isolators are formed of a temperature-responsive material whose wettability changes in response to a temperature.

6. The analysis substrate according to claim 5, wherein the temperature-responsive material is any one of poly(N-isopropylacrylamide), poly(2-alkyl-2-oxazoline), and a polypeptide containing proline.

7. The analysis substrate according to claim 1, wherein the plurality of first isolators are formed of a light-responsive material whose wettability changes in response to light.

8. The analysis substrate according to claim 7, wherein the light-responsive material is any one of diarylethene, an azobenzene-based compound, and a spiropyran-based compound.

9. The analysis substrate according to claim 1, wherein a surface of each of the plurality of capture regions is a surface capable of covalently bonding to the fine particles.

10. The analysis substrate according to claim 1, wherein a surface of each of the plurality of capture regions is a surface treated with citric acid.

11. The analysis substrate according to claim 1, wherein an antibody for capturing the fine particles is immobilized to a surface of each of the plurality of capture regions.

12. The analysis substrate according to claim 1, wherein each of the plurality of capture regions is a region where surface plasmon resonance occurs by irradiation with excitation light.

13. The analysis substrate according to claim 1, wherein the fine particles captured by each of the plurality of capture regions are subjected to multiple stain with a plurality of fluorescent dyes.

14. The analysis substrate according to claim 2, wherein the second isolator is a partition wall separating the plurality of capture regions.

15. An analysis method comprising:
introducing, using an analysis substrate including a plurality of capture regions that capture fine particles, a specimen containing the fine particles into the plurality of capture regions;
separating a surface of the analysis substrate on which the plurality of capture regions are formed into a plurality of first regions each including at least one capture region and provided along a first direction; and
introducing labeling reagents different for each of the plurality of first regions in a state where the surface is separated into the plurality of first regions.

16. The analysis method according to claim 15, wherein each of the labeling reagents different for each of the plurality of first regions contains a combination of different antibodies.

17. The analysis method according to claim 15, wherein each of the labeling reagents different for each of the plurality of first regions contains at least one common antibody.

18. The analysis method according to claim 16, wherein each of the labeling reagents different for each of the plurality of first regions labels the fine particles with fluorescent dyes different for each of the antibodies contained in the labeling reagents.

19. The analysis method according to claim 15, wherein each of the plurality of capture regions is a region where surface plasmon resonance occurs by irradiation with excitation light, and
the analysis method further comprises:
irradiating the plurality of capture regions with the excitation light; and
detecting a fluorescence signal amplified by irradiating the plurality of capture regions with the excitation light and causing the surface plasmon resonance in the plurality of capture regions.

20. The analysis method according to claim 15, wherein the analysis substrate further includes a plurality of first isolators whose wettability changes in response to a stimulus, the plurality of first isolators being provided along the first direction between the capture regions, and
in the separating of the surface of the analysis substrate on which the plurality of capture regions are formed into the plurality of first regions, the surface of the analysis substrate on which the plurality of capture regions are formed is separated into the plurality of first regions by changing the wettability of the plurality of first isolators by applying a stimulus to the plurality of first isolators.

21. The analysis method according to claim 15, wherein a separator for separating the surface of the analysis substrate on which the plurality of capture regions are formed into a plurality of regions is provided, and wherein, in the separating of the surface of the analysis substrate on which the plurality of capture regions are formed into the plurality of first regions, the surface of the analysis substrate on which the plurality of capture regions are formed is separated into the plurality of first regions by arranging the separator on the analysis substrate.

22. The analysis method according to claim 15, further comprising separating the surface of the analysis substrate on which the plurality of capture regions are formed into a plurality of second regions each including at least one capture region and provided along a second direction intersecting with the first direction,
wherein in the introducing the specimens into the plurality of capture regions, the specimens are introduced into the plurality of capture regions by introducing the specimens different for each of the plurality of second regions in a state where the surface is separated into the plurality of second regions.

23. The analysis method according to claim 22, wherein the analysis substrate further includes second isolator whose wettability changes in response to a stimulus, the second isolator being provided along the second direction intersecting with the first direction between the capture regions, and
in the separating of the surface of the analysis substrate on which the plurality of capture regions are formed into the plurality of second regions, the surface of the analysis substrate on which the plurality of capture regions are formed is separated into the plurality of second regions by changing the wettability of the second isolator by applying a stimulus to the second isolator.

24. The analysis method according to claim 22, wherein a separator for separating the surface of the analysis substrate on which the plurality of capture regions are formed into a plurality of regions is provided, and wherein, in the separating of the surface of the analysis substrate on which the plurality of capture regions are formed into the plurality of second regions, the surface of the analysis substrate on which the plurality of capture regions are formed is separated into the plurality of second regions by arranging the separator on the analysis substrate.

25. An analysis system comprising:
an analysis substrate for performing analysis on fine particles; and
an analysis apparatus that performs analysis on the fine particles captured by the analysis substrate,
wherein the analysis substrate includes:
a plurality of capture regions configured to capture the fine particles; and
a plurality of first isolators whose wettability changes in response to a stimulus, the plurality of first isolators being provided along a first direction between the capture regions, and
the analysis apparatus includes:
a stimulus applier configured to apply a stimulus to the plurality of first isolators; and
an imager configured to image the plurality of capture regions.

26. An analysis apparatus comprising:
a stimulus applier configured to apply a stimulus to a plurality of first isolators whose wettability changes in response to a stimulus, the plurality of first isolators being provided between a plurality of capture regions that capture fine particles of an analysis substrate for performing analysis on the fine particles; and
an imager configured to image the plurality of capture regions.
